(19) United States
(12) Patent Application Publication
(10) Pub. No.: US 2023/0190207 A1
(43) Pub. Date: Jun. 22, 2023

(54) INFORMATION PROCESSING METHOD, INFORMATION PROCESSING SYSTEM, TERMINAL, AND NON-TRANSITORY COMPUTER READABLE RECORDING MEDIUM STORING INFORMATION PROCESSING PROGRAM

(71) Applicant: Panasonic Intellectual Property Corporation of America, Torrance, CA (US)

(72) Inventors: Toshiaki TANAKA, Hyogo (JP); Kana MIYATA, Osaka (JP); Yuki MINODA, Hyogo (JP); Ken OHMAE, Tokyo (JP); Yushi NAKAMURA, Osaka (JP); Koh UEHARA, Osaka (JP)

(73) Assignee: Panasonic Intellectual Property Corporation of America, Torrance, CA (US)

(21) Appl. No.: 18/112,936

(22) Filed: Feb. 22, 2023

Related U.S. Application Data
(63) Continuation of application No. PCT/JP2021/030860, filed on Aug. 23, 2021.
(60) Provisional application No. 63/072,311, filed on Aug. 31, 2020.

(30) Foreign Application Priority Data
Aug. 4, 2021 (JP) 2021-128549

(51) Int. Cl. A61B 5/00; A61B 5/01

(52) U.S. Cl. CPC A61B 5/742; A61B 5/01

(57) ABSTRACT

An information processing system includes a first user terminal that acquires body temperature information about a first user, and a second user terminal that presents the body temperature information to a second user. The first user terminal selects an input mode for use in acquiring the body temperature information from among a plurality of input modes each having distinctive reliability for body temperature information to be acquired, acquires the body temperature information in the selected input mode, and transmits the acquired body temperature information and input mode identification information indicating the selected input mode. The second user terminal receives the body temperature information and the input mode identification information, and causes a display part to display the body temperature information in a distinctive display manner in accordance with the input mode identification information.

### TECHNICAL FIELD

This disclosure relates to a technology of presenting body temperature information about a first user to a second user.

### BACKGROUND ART

For instance, Patent Literature 1 discloses that a database server retrieves, upon receipt of: an employee number of an employee who is individually identified; a body temperature measured value; and time information from an information acquisition device, a corresponding employee test file from an external storage device on the basis of the employee number and the time information, and stores the time information and the body temperature measured value received from the information acquisition device in a field of the retrieved employee test file. Besides, Patent Literature 1 discloses arrangement of a plurality of information acquisition devices at respective entering and leaving management doors in a company.

However, the conventional technology faces difficulty in, when presenting measured body temperature information about a first user to a second user, presentation of reliability for the body temperature information to the second user, and thus needs further improvement.

### CITATION LIST

### Patent Literature

Patent Literature 1: Japanese Unexamined Patent Publication No. 2006-293766

### SUMMARY OF INVENTION

This disclosure has been achieved to solve the drawbacks described above, and has an object of providing a technology of, when presenting measured body temperature information about a first user to a second user, presenting reliability for the body temperature information to the second user.

An information processing method according to this disclosure is an information processing method for an information processing system including a first terminal that acquires body temperature information about a first user, and a second user terminal that presents the body temperature information to the user. The information processing method includes: causing the first terminal to: select an input mode for use in acquiring the body temperature information from among a plurality of input modes each having distinctive reliability for body temperature information to be acquired; acquire the body temperature information in the selected input mode; and transmit the acquired body temperature information and input mode identification information indicating the selected input mode; and causing the second terminal to: receive the body temperature information and the input mode identification information; and cause a display part to display the body temperature information in a distinctive display manner in accordance with the input mode identification information.

This disclosure achieves, when presenting measured temperature information about a first user to a second user, presentation of reliability for the body temperature information to the second user.

### BRIEF DESCRIPTION OF DRAWINGS

FIG. 1 is a diagram showing an example of a configuration of a body temperature management system in a first embodiment of the disclosure.

FIG. 2 is a diagram showing an example of a configuration of a first user terminal in the first embodiment of the disclosure.

FIG. 3 is a diagram showing an example of a configuration of a second user terminal in the first embodiment of the disclosure.

FIG. 4 is a flowchart explaining acquisition of body temperature information by the first user terminal in the first embodiment of the disclosure.

FIG. 5 shows an example of a body temperature presentation screen image displayed on the first user terminal in the first embodiment.

FIG. 6 is a flowchart explaining presentation of body temperature information by the second user terminal in the first embodiment of the disclosure.

FIG. 7 shows an example of a body temperature presentation screen image displayed on the second user terminal in the first embodiment

FIG. 8 is a diagram showing an example of a configuration of a first user terminal in a second embodiment of the disclosure.

FIG. 9 is a diagram showing an example of a configuration of a second user terminal in the second embodiment of the disclosure.

FIG. 10 is a flowchart explaining acquisition of body temperature information by the first user terminal in the second embodiment of the disclosure.

FIG. 11 shows an example of a body temperature presentation screen image displayed on the first user terminal in the second embodiment.

FIG. 12 is a flowchart explaining presentation of body temperature information by the second user terminal in the second embodiment of the disclosure.

FIG. 13 shows an example of a body temperature presentation screen image displayed on the second user terminal in the second embodiment.

FIG. 14 is a diagram showing an example of a configuration of a first user terminal in a third embodiment of the disclosure.

FIG. 15 is a flowchart explaining acquisition of body temperature information by the first user terminal in the third embodiment of the disclosure.

FIG. 16 is a diagram showing an example of a configuration of a first user terminal in a fourth embodiment of the disclosure.

FIG. 17 is a first flowchart explaining acquisition of body temperature information by the first user terminal in the fourth embodiment of the disclosure.

FIG. 18 is a second flowchart explaining the acquisition of body temperature information by the first user terminal in the fourth embodiment of the disclosure.

FIG. 19 shows an example of a good health condition certificate presentation screen image displayed on the first user terminal in the fourth embodiment.

FIG. 20 shows an example of a good health condition certificate presentation screen image displayed on a first user terminal in a modification of the fourth embodiment.

FIG. 21 is a diagram showing an example of a configuration of a first user terminal in a fifth embodiment of the disclosure.

FIG. 22 is a first flowchart explaining acquisition of body temperature information by the first user terminal in the fifth embodiment of the disclosure.

FIG. 23 is a second flowchart explaining the acquisition of body temperature information by the first user terminal in the fifth embodiment of the disclosure.

FIG. 24 shows an example of a preferential virus test certificate presentation screen image displayed on the first user terminal in the fifth embodiment.

FIG. 25 shows an example of a first reliability level-preferential virus test certificate presentation screen image displayed on a first user terminal in a modification of the fifth embodiment.

FIG. 26 shows an example of a second reliability level-preferential virus test certificate presentation screen image displayed on the first user terminal in the modification of the fifth embodiment.

FIG. 27 shows an example of a third reliability level-preferential virus test certificate presentation screen image displayed on the first user terminal in the modification of the fifth embodiment.

### DESCRIPTION OF EMBODIMENTS

Knowledge forming the basis of the present disclosure

In the aforementioned conventional technology, an information acquisition device is arranged at an entering and leaving management door in a company. In this regard, it costs a lot to arrange the information acquisition device. Under the circumstances, it is considered to acquire a body temperature of a user through a terminal, such as a smartphone, owned by the user. In this case, the terminal receives a manual input, by the user, of a body temperature measured with a thermometer, and transmits the received body temperature to a server.

However, the user may intentionally input a wrong body temperature, which leads to lower reliability for the input body temperature information. In this respect, when measured body temperature information about a first user is presented to a second user, the second user finds it difficult to grasp a reliability level for the presented body temperature information.

To solve the drawbacks described above, an information processing method according to an aspect of the disclosure is an information processing method for an information processing system including a first terminal that acquires body temperature information about a first user and a second terminal that presents the body temperature information to a second user. The information processing method includes: causing the first terminal to: select an input mode for use in acquiring the body temperature information from among a plurality of input modes each having distinctive reliability for body temperature information to be acquired; acquire the body temperature information in the selected input mode; and transmit the acquired body temperature information and input mode identification information indicating the selected input mode; and causing the second terminal to: receive the body temperature information and the input mode identification information; and cause a display part to display the body temperature information in a distinctive display manner in accordance with the input mode identification information.

According to this configuration, input mode identification information indicating an input mode for use in acquiring body temperature information among the input modes each having distinctive reliability for body temperature information to be acquired is received together with the body temperature information, and the body temperature information is displayed on the display part in a distinctive display manner in accordance with the input mode identification information. This achieves, when presenting measured temperature information about the first user to the second user, presentation of reliability for the body temperature information to the second user.

In the information processing method, the input modes may include a first input mode of acquiring the body temperature information from a thermometer, which measures a body temperature of the first user, through communication therewith and a second input mode of acquiring the body temperature information from an input receiving part which receives a manual input of the body temperature by the user. In the displaying of the body temperature information, the input mode indicated by the input mode identification information may be determined as either the first input mode or the second input mode. The body temperature information may be displayed on the display part in a first display manner when the input mode is determined as the first input mode, and the body temperature information may be displayed on the display part in a second display manner which is different from the first display manner when the input mode is determined as the second input mode.

The body temperature information acquired in the first input mode is more difficult to be falsified than the body temperature information acquired in the second input mode. In this respect, reliability for the body temperature information acquired in the first input mode is higher than reliability for the body temperature information acquired in the second input mode. It is seen from this perspective that body temperature information is displayed in a distinctive manner between a case where a selected input mode indicates the first input mode of acquiring body temperature information from the thermometer, which measures a body temperature of the first user, through communication therewith and a case where the selected input mode indicates the second input mode of acquiring body temperature information from the input receiving part which receives a manual input of the body temperature by the first user. Therefore, the second user can easily confirm the reliability for the displayed body temperature information by seeing the displayed body temperature information.

The information processing method may cause the first terminal to: further generate a good health condition certificate proving that the first user has a good health condition when the selected input mode indicates the first input mode and the acquired body temperature information falls below a predetermined value; and further cause a display part to display the generated good health condition certificate.

According to this configuration, a good health condition certificate proving that the first user has a good health condition is generated when the body temperature information acquired in the first input mode having higher reliability than the second input mode falls below the predetermined value, and therefore, the first user can prove a good health condition thereof with the good health condition certificate.

In the information processing method, in the generating of the good health condition certificate, the good health condition certificate may be generated when the selected input mode continuously indicates the first input mode throughout a predetermined period and the acquired body temperature information continuously falls below the predetermined value throughout the predetermined period.

According to this configuration, a good health condition certificate proving that the first user has a good health condition is generated when body temperature information is continuously acquired in the first input mode having higher reliability than the second input mode throughout the predetermined period and the acquired body temperature information continuously falls below the predetermined value throughout the predetermined period, and therefore, the first user can prove a good health condition thereof with the good health condition certificate.

The information processing method may cause the first terminal to: further generate a preferential virus test certificate proving that the first user is preferentially prompted to receive a virus test when the selected input mode continuously indicates the first input mode throughout a predetermined period and the acquired body temperature information continuously indicates the predetermined value or higher throughout the predetermined period; and cause the display part to further display the generated preferential virus test certificate.

According to this configuration, a preferential virus test certificate proving that the first user is preferentially prompted to receive a virus test when body temperature information is continuously acquired in the first input mode having higher reliability than the second input mode throughout the predetermined period and the acquired body temperature information continuously indicates the predetermined value or higher throughout the predetermined period, and therefore, the first user can preferentially receive the virus test owing to the preferential virus test certificate.

In the information processing method, in the displaying of the body temperature information, a sign having a distinctive shape in accordance with the input mode identification information may be displayed in the vicinity of the body temperature information, a sign having a distinctive color in accordance with the input mode identification information may be displayed in the vicinity of the body temperature information, the body information may be displayed in a distinctive color in accordance with the input mode identification information, or a name of the input mode indicated by the input mode identification information may be displayed in the vicinity of the body temperature information.

According to this configuration, the second user can easily recognize the reliability for the body temperature information about the first user by confirming the shape of the sign displayed in the vicinity of the body temperature information, the color of the sign displayed in the vicinity of the body temperature information, the color of the body temperature information, or a name of an input mode displayed in the vicinity of the body temperature information.

In the information processing method, the first terminal may cause the display part to display the body temperature information in a distinctive display manner in accordance with the selected input mode.

According to this configuration, the body temperature information is displayed on the display part in the first terminal in the distinctive display manner in accordance with the selected input mode, and therefore, the reliability for the body temperature information is presentable to the first user. Besides, the first user can easily recognize the reliability for the body temperature information about the first user by confirming the displayed body temperature information.

In the information processing method, in the acquiring of the body temperature information, measurement device information may be acquired together with the body temperature information, the measurement device information including at least one of information indicating whether a body temperature is measured in a predictive measurement manner or an actual measurement manner, information indicating whether the body temperature is measured in a contact manner or a contactless manner, and measured portion information indicating a body portion at which the body temperature has been measured. In the transmitting of the body temperature information and the input mode identification information, the acquired measurement device information may be transmitted together with the body temperature information and the input mode identification information. In the receiving of the body temperature information and the input mode identification information, the measurement device information may be received together with the body temperature information and the input mode identification information. In the displaying of the body temperature information, the body temperature information may be displayed on the display part in a distinctive display manner in accordance with the input mode identification information and the measurement device information.

Reliability for the body temperature information measured in the actual measurement manner is higher than reliability for the body temperature information measured in the predictive measurement manner. Besides, reliability for the body temperature information measured in the contact manner is higher than reliability for the body temperature information measured in the contactless manner. Moreover, the reliability for the body temperature information differs depending on a body portion to be measured. From these perspectives, the body temperature information measured in the predictive measurement manner is displayed distinctively from the body temperature information measured in the actual measurement manner, and therefore, the reliability for the body temperature about the first user is presentable to the second user. Furthermore, the body temperature information measured in the contact manner is displayed distinctively from the body temperature information measured in the contactless manner, and therefore, the reliability for the body temperature information about the first user is presentable to the second user. In addition, the body temperature information is displayed in a distinctive display manner in accordance with a body portion at which the body temperature has been measured, and thus, reliability for the body temperature information about the first user is presentable to the second user.

In the information processing method, in the acquiring of the body temperature information, device identification information for identifying a thermometer which measures a body temperature of the first user may be acquired together with the body temperature information. In the transmitting of the body temperature information and the input mode identification information, the acquired device identification information may be transmitted together with the body temperature information and the input mode identification information. In the receiving of the body temperature information and the input mode identification information, measurement device information may be received together with the body temperature information and the input mode identification information, the measurement device information being specified on the basis of the device identification information and including at least one of information indicating whether the body temperature is measured in a predictive measurement manner or an actual measurement manner, information indicating whether the body temperature is measured in a contact manner or a contactless manner, and measured portion information indicating a body portion at which the body temperature has been measured. In the displaying of the body temperature information, the body temperature information may be displayed on the display part in a distinctive display manner in accordance with the input mode identification information and the measurement device information.

According to this disclosure, the body temperature information measured in the predictive measurement manner is displayed distinctively from the body temperature information measured in the actual measurement manner, and therefore, reliability for the body temperature information about the first user is presentable to the second user. Furthermore, the body temperature information measured in the contact manner is displayed distinctively from the body temperature information measured in the contactless manner, and therefore, the reliability for the body temperature information about the first user is presentable to the second user. In addition, the body temperature information is displayed in a distinctive display manner in accordance with a body portion at which the body temperature has been measured, and thus, reliability for the body temperature information about the first user is presentable to the second user.

The device identification information having a data amount smaller than that of the measurement device information is transmitted from the first terminal, and therefore, reduction in a data amount of communication is achieved and a shorter time for the communication is attained.

Moreover, the disclosure can be realized as: the information processing method executing the above-described distinctive ways; and an information processing system including each distinctive feature corresponding to the distinctive ways executed by the information processing method. From these perspectives, the same advantageous effects as those of the information processing method are achievable in the following other aspects.

An information processing system according to another aspect of the disclosure includes: a first terminal that acquires body temperature information about a first user; and a second terminal that presents the body temperature information to a second user. The first terminal includes: a selection part that selects an input mode for use in acquiring the body temperature information from among a plurality of input modes each having distinctive reliability for body temperature information to be acquired; an acquisition part that acquires the body temperature information in the selected input mode; and a transmission part that transmits the acquired body temperature information and input mode identification information indicating the selected input mode. The second terminal includes: a receiving part that receives the body temperature information and the input mode identification information; and a display control part that causes a display part to display the body temperature information in a distinctive display manner in accordance with the input mode identification information.

An information processing method according to further another aspect of the disclosure is an information processing method for a second terminal that presents body temperature information about a first user acquired by a first terminal to a second user. The information processing method includes: receiving, from the first terminal which: selects an input mode for use in acquiring the body temperature information from among a plurality of input modes each having distinctive reliability for body temperature information to be acquired; acquires the body temperature information in the selected input mode; and transmits the acquired body temperature information and input mode identification information indicating the input mode, the body temperature information and the input mode identification information; and causing a display part to display the body temperature information in a distinctive display manner in accordance with the input mode identification information.

According to this configuration, input mode identification information indicating an input mode for use in acquiring the body temperature information among the input modes each having distinctive reliability for body temperature information to be acquired is received together with the body temperature information, and the body temperature information is displayed on the display part in a distinctive display manner in accordance with the input mode identification information. This achieves, when presenting measured temperature information about the first user to the second user, presentation of reliability for the body temperature information to the second user.

Moreover, the disclosure can be realized as: the information processing method executing the above-described distinctive ways; and a terminal including each distinctive feature corresponding to the distinctive ways executed by the information processing method.

Additionally, the disclosure can be realized by a computer program causing a computer to execute the distinctive ways included in the information processing method. From these perspectives, the same advantageous effects as those of the information processing method are achievable in the following other aspects.

A terminal according to another aspect of the disclosure is a terminal that presents body temperature information about a first user acquired by another terminal to a second user. The terminal includes: a receiving part that receives, from the another terminal which: selects an input mode for use in acquiring the body temperature information from among a plurality of input modes each having distinctive reliability for body temperature information to be acquired; acquires the body temperature information in accordance with the selected input mode; and transmits the acquired body temperature information and input mode identification information indicating the selected input mode, the body temperature information and the input mode identification information; and a display control part that causes a display part to display the body temperature information in a distinctive display manner in accordance with the input mode identification information.

A non-transitory computer readable recording medium according to another aspect of the disclosure stores an information processing program for presenting body temperature information about a first user acquired by another terminal to a second user. The information processing program including: causing a computer to execute: receiving, from the another terminal which: selects an input mode for use in acquiring the body temperature information from among a plurality of input modes each having distinctive reliability for body temperature information to be acquired; acquires the body temperature information in the selected input mode; and transmits the acquired body temperature information and input mode identification information indicating the selected input mode, the body temperature information and the input mode identification information; and causing a display part to display the body temperature information in a distinctive display manner in accordance with the input mode identification information.

Embodiments of this disclosure will be described with reference to the accompanying drawings. It should be noted that each of the following embodiments illustrates one example of the disclosure, and does not delimit the technical scope of the disclosure.

### First Embodiment

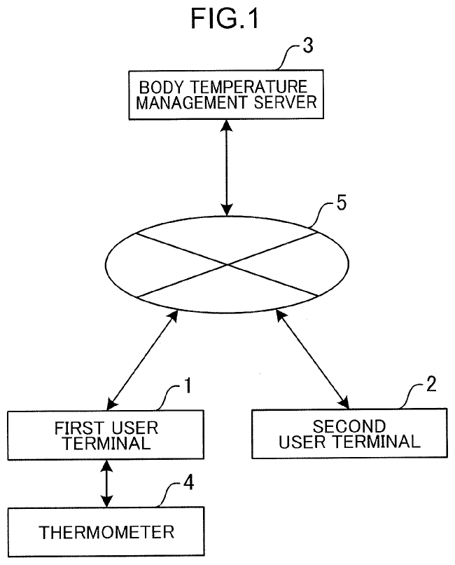

FIG. 1 is a diagram showing an example of a configuration of a body temperature management system in a first embodiment of the disclosure.

The body temperature management system shown in FIG. 1 includes a first user terminal 1, a second user terminal 2, and a body temperature management server 3.

The first user terminal 1 is, for example, a smartphone, a personal computer, or a tablet-type computer, and is used by a first user. The first user is, for example, an employee of a certain company.

The first user terminal 1 acquires body temperature information about the first user, for example, once a day. The first user terminal 1 has a plurality of input modes each having distinctive reliability for body temperature information to be acquired. The input modes include a communication input mode (first input mode) of acquiring the body temperature information from a thermometer 4, which measures a body temperature of the first user, through communication therewith, and a manual input mode (second input mode) of acquiring the body temperature information from an input receiving part which receives a manual input of the body temperature by the first user. The first user terminal 1 receives a selection of an input mode for use in acquiring the body temperature information by the first user.

When the communication input mode is selected, the first user terminal 1 acquires body temperature information about the first user from the thermometer 4 communicably connected to the first user terminal 1.

The thermometer 4 is communicably connected to the first user terminal 1. The thermometer 4 transmits the measured body temperature information about the first user to the first user terminal 1 through a near field communication.

The first user terminal 1 is communicably connected to the thermometer 4 by using a predetermined near field communication standard. The predetermined near field communication standard is, for example, the Bluetooth (registered trademark). The first user terminal 1 and the thermometer 4 are paired with each other in advance.

By contrast, when the manual input mode is selected, the first user terminal 1 receives a manual input of a body temperature by the first user. The first user measures a body temperature thereof by using a thermometer, and inputs the measured body temperature to the first user terminal 1. When the thermometer 4 has a communication function, the thermometer 4 can automatically transmit the measured body temperature information to the first user terminal 1. Contrarily, when the thermometer 4 has no communication function, the thermometer 4 cannot transmit the measured body temperature information to the first user terminal 1. Hence, the first user needs to manually input a result of the measurement displayed on the thermometer 4 to the first user terminal 1.

The body temperature information acquired from the thermometer 4 through the communication therewith is difficult to be falsified, but the body temperature information acquired through the manual input is easily falsified. Therefore, reliability for the body temperature information acquired through the communication is higher than reliability for the body temperature information acquired through the manual input.

The first user terminal 1 transmits, to the body temperature management server 3, the acquired body temperature information about the first user and input mode identification information indicating the selected input mode. The input mode identification information identifies whether the input mode indicates the communication input mode or the manual input mode.

The second user terminal 2 is, for example, a smartphone, a personal computer, or a tablet-type computer, and is used by a second user. The second user is, for example, an employee of the same company as the first user, and serves as a manager who manages the first user. Specifically, the first user and the second user belong to the same group (department) of the same company, and the second user is a senior and the first user is a junior.

The second user terminal 2 presents the body temperature information about the first user to the second user. The second user terminal 2 receives the body temperature information about the first user and the input mode identification information from the body temperature management server 3, and displays the received body temperature information about the first user in a distinctive display manner in accordance with the input mode identification information. The second user terminal 2 may acquire body temperature information about the second user and transmit the acquired body temperature information of the second user to the body temperature management server 3.

Although the body temperature management system includes the single first user terminal 1 and the single second user terminal 2 in the first embodiment, this disclosure is not particularly limited thereto, and the system may include a plurality of first user terminals 1 and a plurality of second user terminals 2.

The body temperature management server 3 includes, for example, a cloud server, and is communicably connected to the first user terminal 1 and the second user terminal 2 via a network 5. The network 5 includes, for example, the internet. The body temperature management server 3 manages the body temperature information about each of the first user and the second user. The body temperature management server 3 receives the body temperature information about the first user and the input mode identification information from the first user terminal 1. The body temperature management server 3 transmits the received body temperature information about the first user and input mode information to the second user terminal 2.

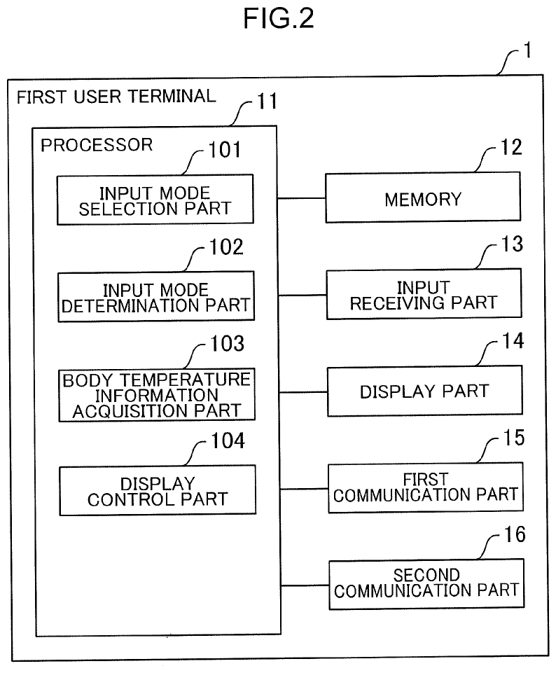

FIG. 2 is a diagram showing an example of a configuration of the first user terminal 1 in the first embodiment of the disclosure.

The first user terminal 1 shown in FIG. 2 includes a processor 11, a memory 12, an input receiving part 13, a display part 14, a first communication part 15, and a second communication part 16. The first user terminal 1 serves as an example of each of a first terminal and another terminal.

The processor 11 includes, for example, a CPU (central processing unit). The processor 11 realizes an input mode selection part 101, an input mode determination part 102, a body temperature acquisition part 103, and a display control part 104.

For instance, the memory 12 includes a storage device, such as a RAM (Random Access Memory), an SSD (Solid State Drive), or a flash memory, for storing various kinds of information. The memory 12 stores various kinds of information.

The input receiving part 13 includes, for example, a touch screen, a keyboard, or a mouse. The display part 14 includes, for example, a liquid crystal display device. The first communication part 15 communicates with the thermometer 4 in accordance with a predetermined near field communication standard. The second communication part 16 communicates with the body temperature management server 3 via the network 5.

The input mode selection part 101 selects an input mode for use in acquiring body temperature information from among the input modes each having distinctive reliability for body temperature information to be acquired. The input mode selection part 101 causes the display part 14 to display an input mode selection screen image for receiving a selection of an input mode, i.e., either the communication input mode or the manual input mode.

The input receiving part 13 receives a selection of an input mode for use in acquiring the body temperature information by the first user from among the input modes. The first user selects an input mode from the communication input mode and the manual input mode on the input mode selection screen image displayed on the display part 14.

The input mode determination part 102 determines which input mode is selected by the input mode selection part 101, i.e., either the communication input mode or the manual input mode.

The body temperature acquisition part 103 acquires body temperature information in the input mode selected by the input mode selection part 101.

When the input mode selection part 101 selects the communication input mode, the body temperature acquisition part 103 acquires the body temperature information from the thermometer 4 through communication therewith via the first communication part 15. At this time, the first communication part 15 establishes the communication with the thermometer 4 and receives the body temperature information from the thermometer 4.

When the input mode selection part 101 selects the manual input mode, the body temperature acquisition part 103 receives, from the input receiving part 13, the body temperature information manually input by the first user. At this time, the body temperature acquisition part 103 causes the display part 14 to display a body temperature input screen image for receiving a manual input of a body temperature by the first user. The input receiving part 13 receives the manual input of the body temperature by the first user on the body temperature input screen image displayed on the display part 14.

The display control part 104 causes the display part 14 to display the body temperature information in a distinctive display manner in accordance with the input mode selected by the input mode selection part 101. The display control part 104 executes displaying of a sign having a distinctive shape in accordance with the input mode in the vicinity of the body temperature information. The display control part 104 may execute displaying of a sign having a distinctive color in accordance with the input mode in the vicinity of the body temperature information, may execute displaying of the body temperature information in a distinctive color in accordance with the input mode, or may execute displaying of a name of the input mode in the vicinity of the body temperature information.

When the input mode is determined as the communication input mode, the display control part 104 causes the display part 14 to display the body temperature information in a first display manner in accordance with the communication input mode. When the input mode is determined as the manual input mode, the display control part 104 causes the display part 14 to display the body temperature information in a second display manner, which is different from the first display manner, in accordance with the manual input mode.

The second communication part 16 transmits the body temperature information acquired by the body temperature acquisition part 103 and the input mode identification information indicating the input mode selected by the input mode selection part 101. The second communication part 16 transmits the body temperature information and the input mode identification information to the body temperature management server 3.

The body temperature management server 3 receives the body temperature information and the input mode identification information transmitted from the first user terminal 1. The body temperature management server 3 stores user information for each of the users. Examples of the user information include a user ID specifying a user, a name of the user, a residence of the user, an age of the user, and a group ID specifying a group to which the user belongs. The body temperature management server 3 further stores the group ID in association with the second user terminal 2 of the manager (second user) in the group in advance. The body temperature management server 3 determines the second user terminal 2 of the second user associated with the group ID of the group to which the first user belongs to be a destination of the body temperature information and the input mode identification information. The body temperature management server 3 transmits the received body temperature information about the first user and input mode identification information to the second user terminal 2.

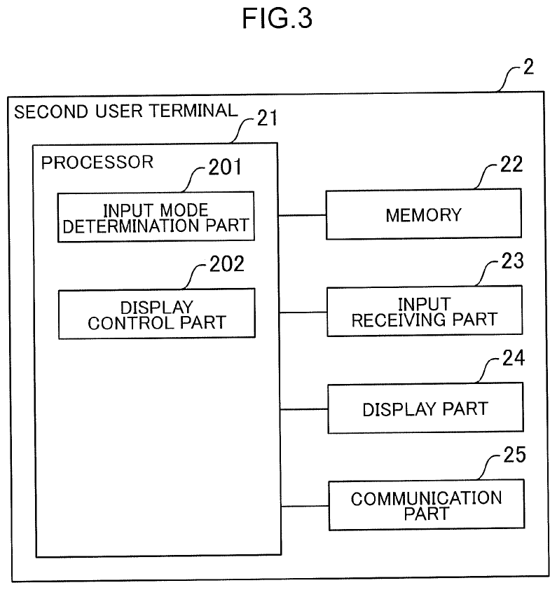

FIG. 3 is a diagram showing an example of a configuration of the second user terminal 2 in the first embodiment of the disclosure.

The second user terminal 2 shown in FIG. 3 includes a processor 21, a memory 22, an input receiving part 23, a display part 24, and a communication part 25. The second user terminal 2 serves as an example of a second terminal and a terminal.

The processor 21 includes, for example, a CPU. The processor 21 realizes an input mode determination part 201 and a display control part 202.

For instance, the memory 22 includes a storage device, such as a RAM, an SSD, or a flash memory, for storing various kinds of information. The memory 22 stores various kinds of information.

The input receiving part 23 includes, for example, a touch screen, a keyboard, or a mouse. The display part 24 includes, for example, a liquid crystal display device. The communication part 25 communicates with the body temperature management server 3 via the network 5.

The communication part 25 receives body temperature information and input mode identification information. The communication part 25 receives the body temperature information and the input mode identification information transmitted from the body temperature management server 3.

The input mode determination part 201 determines which input mode is indicated by the input mode identification information indicates, i.e., either the communication input mode or the manual input mode.

The display control part 202 causes the display part 24 to display the body temperature information in a distinctive display manner in accordance with the input mode identification information. The display control part 202 executes displaying of a sign having a distinctive shape in accordance with the input mode identification information in the vicinity of the body temperature information. The display control part 202 may execute displaying of a sign having a distinctive color in accordance with the input mode identification information in the vicinity of the body temperature information, may execute displaying of the body temperature information in a distinctive color in accordance with the input mode identification information, or may execute displaying of a name of the input mode indicated by the input mode identification information in the vicinity of the body temperature information.

When the input mode is determined as the communication input mode, the display control part 202 causes the display part 24 to display the body temperature information in a first display manner in accordance with the communication input mode. When the input mode is determined as the manual input mode, the display control part 202 causes the display part 24 to display the body temperature information in a second display manner, which is different from the first display manner, in accordance with the manual input mode.

Next, acquisition of body temperature information by the first user terminal 1 in the first embodiment of the disclosure will be described.

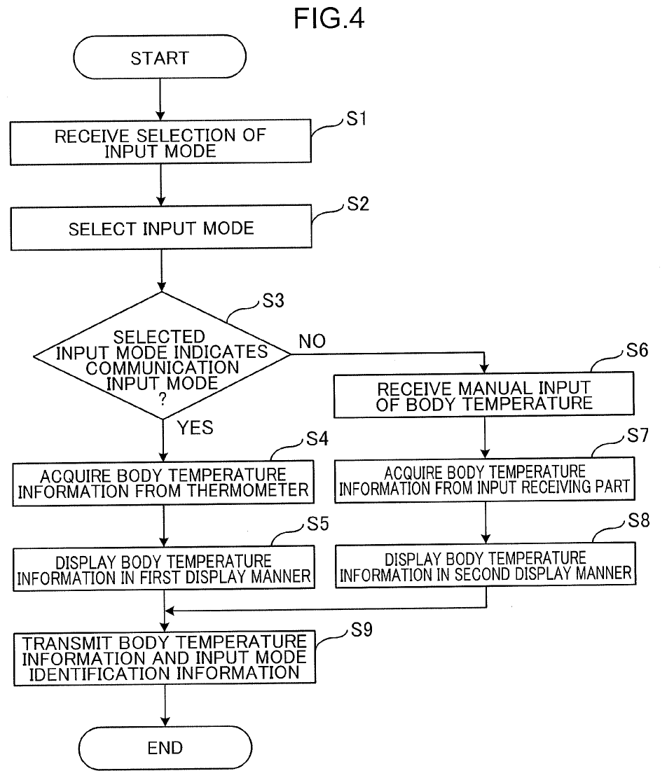

FIG. 4 is a flowchart explaining the acquisition of body temperature information by the first user terminal 1 in the first embodiment of the disclosure.

First, in step S1, the input receiving part 13 receives a selection of an input mode for use in acquiring body temperature information by the first user from the communication input mode and the manual input mode. The input mode selection part 101 causes the display part 14 to display an input mode selection screen image for receiving a selection of an input mode, i.e., either the communication input mode or the manual input mode. The input mode selection screen image has a first button for receiving the selection of the communication input mode and a second button for receiving the selection of the manual input mode. The communication input mode is selected in response to touching of the first button by the first user, and the manual input mode is selected in response to touching of the second button by the first user.

Subsequently, in step S2, the input mode selection part 101 selects an input mode for use in acquiring body temperature information from the communication input mode and the manual input mode. The input mode selection part 101 selects the input mode received by the input receiving part 13.

Next, in step S3, when the input mode determination part 102 determines whether the input mode selected by the input mode selection part 101 indicates the communication input mode.

Here, when the selected input mode is determined as the communication input mode (YES in step S3), the body temperature acquisition part 103 acquires, in step S4, the body temperature information from the thermometer 4 through communication therewith via the first communication part 15. At this time, the first communication part 15 establishes a communication with the thermometer 4 to receive the body temperature information from the thermometer 4 through a near field communication.

Then, in step S5, the display control part 104 causes the display part 14 to display the body temperature information in the first display manner in accordance with the communication input mode. The display part 14 displays the body temperature information in the first display manner in response to an instruction from the display control part 104.

By contrast, when the selected input mode is not determined as the communication input mode, that is, when the selected input mode is determined as the manual input mode (NO in step S3), the input receiving part 13 receives, in step S6, a manual input of a body temperature by the first user. The body temperature acquisition part 103 causes the display part 14 to display a body temperature input screen image for receiving the manual input of the body temperature by the first user. The body temperature input screen image has an input section for receiving the manual input of the body temperature. The first user inputs the body temperature thereof measured by the first user in the input section.

The body temperature input screen image may have a default value (e.g., 36.5° C.) for a body temperature, a first button for increasing the default value, and a second button for decreasing the default value. The displayed default value is increased in response to touching of the first button by the first user, and the displayed default value is decreased in response to touching of the second button by the first user. The input receiving part 13 may receive a manual input of the body temperature by increasing or decreasing the default value for the body temperature displayed on the body temperature input screen image.

Next, in step S7, the body temperature acquisition part 103 acquires, from the input receiving part 13, the body temperature information manually input by the first user.

Subsequently, in step S8, the display control part 104 causes the display part 14 to display the body temperature information in the second display manner in accordance with the manual input mode. The display part 14 displays the body temperature information in the second display manner in response to an instruction from the display control part 104.

Then, in step S9, the second communication part 16 transmits the body temperature information acquired by the body temperature acquisition part 103 and the input mode identification information indicating the input mode selected by the input mode selection part 101.

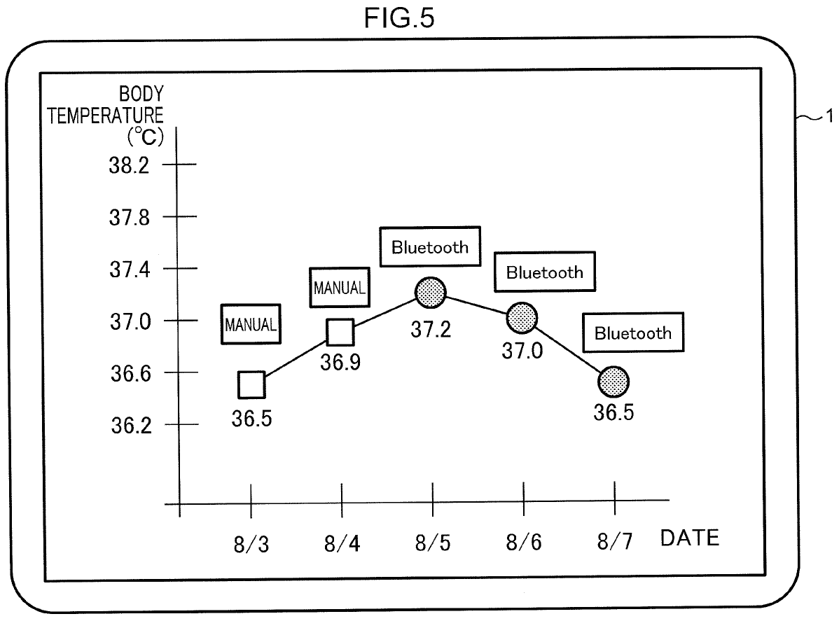

FIG. 5 shows an example of a body temperature presentation screen image displayed on the first user terminal 1 in the first embodiment.

As shown in FIG. 5, the display control part 104 executes the displaying of the body temperature information in the first display manner in accordance with the communication input mode when the body temperature information is acquired in the communication input mode, and executes the displaying of the body temperature information in the second display manner in accordance with the manual input mode when the body temperature information is acquired in the manual input mode. The body temperature presentation screen image in FIG. 5 shows a history of body temperature information in a period from August 3^{rd} to August 7^{th} on a graph having a horizontal axis denoting a date and a vertical axis denoting a body temperature. The body temperature information on August 3^{rd} and August 4^{th} is acquired in the manual input mode, and the body temperature information in a period from August 5^{th} to August 7^{th} is acquired in the communication input mode.

When the body temperature information is acquired in the communication input mode, the display control part 104 executes displaying of a circular sign at a coordinate position corresponding to a date and a body temperature on the graph. The display control part 104 further executes the displaying of the circular sign in a red color. Besides, the display control part 104 executes displaying of a body temperature in a red color under the circular sign. Moreover, the display control part 104 executes displaying of a label indicating acquisition in the communication input mode above the circular sign. Examples of the label include the word “Bluetooth (registered trademark)”.

In addition, when the body temperature information is acquired in the manual input mode, the display control part 104 executes displaying of a square sign at a coordinate position corresponding to a date and a temperature on the graph. The display control part 104 further executes the displaying of the square sign in a white color. Besides, the display control part 104 executes displaying of a body temperature in a black color under the square sign. Moreover, the display control part 104 executes displaying of a label indicating acquisition in the communication input mode above the square sign. Examples of the label include the word “manual”.

Here, each of the shape and the color of each sign is a mere example, and another shape and another color may be adopted. Also, each color for each body temperature to be displayed under the relevant sign is a mere example, and another color may be adopted. Besides, the word or term of the label above the sign shows a mere example, and another word or term may be adopted. Further, the display control part 104 may change only the shape of the sign indicating the body temperature for each of the communication input mode and the manual input mode. Moreover, the display control part 104 may change only the color of the sign indicating the body temperature for each of the communication input mode and the manual input mode. In addition, the display control part 104 may avoid the displaying of the body temperature under the sign and avoid the displaying of the label above the sign.

Furthermore, although the display control part 104 executes the displaying of the history of body temperature information on the graph, this disclosure is not particularly limited thereto, and the display control part may execute displaying of only body temperature information measured on an exact date.

As described heretofore, the body temperature information acquired in the communication input mode is displayed distinctively from the body temperature information acquired in the manual input mode, and therefore, reliability for the body temperature information is presentable to the first user. Further, the first user can easily confirm the reliability for the body temperature information.

Next, presentation of body temperature information by the second user terminal 2 in the first embodiment of the disclosure will be described.

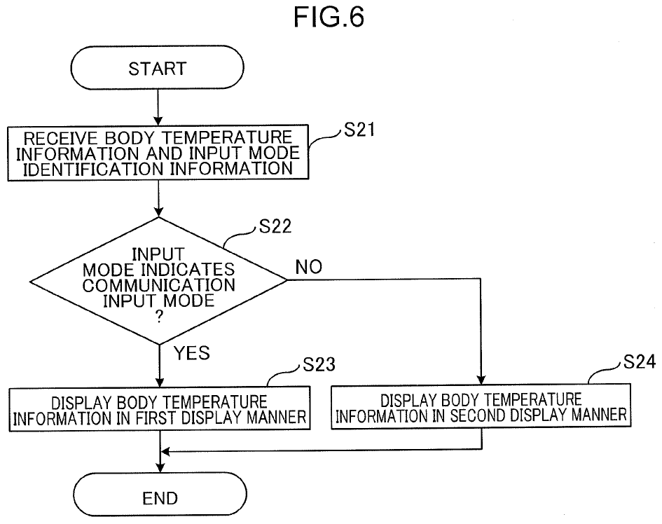

FIG. 6 is a flowchart explaining the presentation of body temperature information by the second user terminal 2 in the first embodiment of the disclosure.

First, in step S21, the communication part 25 receives body temperature information and input mode identification information.

Next, in step S22, the input mode determination part 201 determines whether an input mode indicated by the input mode identification information received by the communication part 25 indicates the communication input mode.

Here, when the input mode is determined as the communication input mode (YES in step S22), the display control part 202 causes, in step S23, the display part 24 to display the body temperature information in the first display manner in accordance with the communication input mode. The display part 24 displays the body temperature information in the first display manner in response to an instruction from the display control part 202.

By contrast, when the input mode is not determined as the communication input mode, that is, when the input mode is determined as the manual input mode (NO in step S22), the display control part 202 causes, in step S24, the display part 24 to display the body temperature information in the second display manner in accordance with the manual input mode. The display part 24 displays the body temperature information in the second display manner in response to an instruction from the display control part 202.

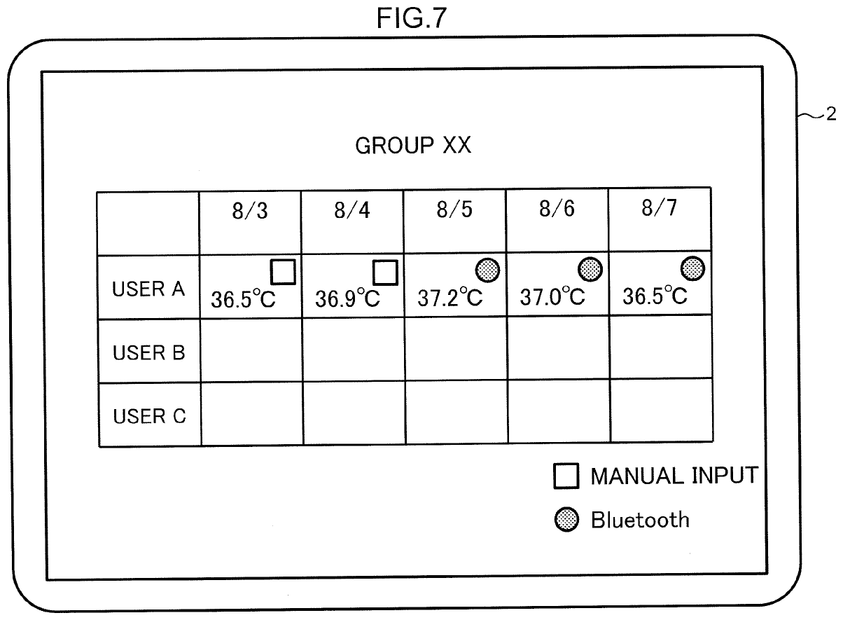

FIG. 7 shows an example of a body temperature presentation screen image displayed on the second user terminal 2 in the first embodiment.

As shown in FIG. 7, the display control part 202 executes the displaying of the body temperature information in the first display manner in accordance with the communication input mode when the body temperature information is acquired in the communication input mode, and executes the displaying of the body temperature information in the second display manner in accordance with the manual input mode when the body temperature information is acquired in the manual input mode. The body temperature presentation screen image in FIG. 7 shows a history of body temperature information about each first user in a period from August 3^{rd} to August 7^{th} on a table associating a name of a member belonging to a group managed by the second user with a relevant date. The body temperature information about a user A on August 3^{rd} and August 4^{th} is acquired in the manual input mode, and the body temperature information about the user A in a period from August 5^{th} to August 7^{th} is acquired in the communication input mode.

When the body temperature information is acquired in the communication input mode, the display control part 202 executes displaying of a body temperature at a position corresponding to a user name and a date on the table, and executes displaying of a circular sign at an upper right position of the body temperature. The display control part 202 further executes the displaying of the circular sign in a red color. Besides, the display control part 202 executes the displaying of the body temperature in a red color.

When the body temperature information is acquired in the manual input mode, the display control part 202 executes displaying of a body temperature at a position corresponding to a user name and a date on the table, and executes displaying of a square sign at an upper right position of the body temperature. The display control part 202 further executes the displaying of the square sign in a white color. Besides, the display control part 202 executes the displaying of the body temperature in a black color.

Here, each of the shape and the color of each sign is a mere example, and another shape and another color may be adopted. Also, each color for each body temperature to be displayed is a mere example, and another color may be adopted. Further, the display control part 202 may change only the shape of the sign for each of the communication input mode and the manual input mode. Moreover, the display control part 202 may change only the color of the sign for each of the communication input mode and the manual input mode.

Furthermore, although the display control part 202 executes the displaying of the history of body temperature information on the table, this disclosure is not particularly limited thereto, and the display control part may execute displaying of only body temperature information about the first user measured on an exact date. In addition, the display control part 202 may execute the displaying of the history of body temperature information about the first user on the graph shown in FIG. 5.

As described heretofore, the body temperature information acquired in the communication input mode is displayed distinctively from the body temperature information acquired in the manual input mode, and therefore, reliability for the body temperature information about the first user is presentable to the second user. Further, the second user can easily confirm the reliability for the body temperature information about the first user.

### Second Embodiment

In the first embodiment, the first user terminal 1 acquires body temperature information from the thermometer 4. By contrast, in a second embodiment, a first user terminal 1A acquires measurement device information, in addition to body temperature information, from a thermometer 4. The measurement device information includes at least one of information indicating whether a body temperature is measured in a predictive measurement manner or an actual measurement manner, information indicating whether the body temperature is measured in a contact manner or a contactless manner, and measured portion information indicating a body portion at which the body temperature has been measured.

The thermometer 4 includes a predictive measurement thermometer and an actual measurement thermometer. The actual measurement thermometer executes measurement until a measured value of a body temperature stops rising, and outputs a result of the measurement of the body temperature when the measured value of the body temperature stops rising. For instance, the measured value of the body temperature stops rising after around ten minutes in a case where an armpit is adopted as the measured portion, and the measured value of the body temperature stops rising after around five minutes in a case where an inside of a mouth is adopted as the measured portion. By contrast, the predictive measurement thermometer predicts a body temperature when a measured value of the body temperature stops rising by analyzing a temperature change in measured values until a lapse of a predetermined time from a start of the measurement, and outputs the predicted body temperature as a result of the measurement. For instance, in the case where the armpit is adopted as the measured portion, the predictive measurement thermometer predicts a body temperature after ten minutes at which a measured value of the body temperature stops rising by analyzing a temperature change in measured temperature values in fifteen seconds from a start of the measurement. Moreover, for example, in the case where the inside of the mouth is adopted as the measured portion, the predictive measurement thermometer predicts a body temperature after five minutes at which a measured value of the body temperature stops rising by analyzing a temperature change in measured values in fifteen seconds from a start of the measurement. Generally, reliability for the body temperature measured by the actual measurement thermometer is higher than reliability for the body temperature measured by the predictive measurement thermometer.

The thermometer 4 further includes a contact-type thermometer and a contactless-type thermometer. The contact-type thermometer measures a body temperature by bringing a sensor thereof into contact with a specific body portion of a person. Examples of the sensor include a thermistor. The contactless-type thermometer measures an amount of infrared rays emitted from a surface of a body (e.g., forehead) of a person, and convers the measured amount of infrared rays into a body temperature. Generally, reliability for the body temperature measured by the contact-type thermometer is higher than reliability for the body temperature measured by the contactless-type thermometer.

Moreover, the thermometer 4 can measure a temperature of a specific body portion as a body temperature. Examples of the body portion to be measured include a lower section of a tongue, an armpit, an inside (eardrum) of a ear, and a forehead. Generally, reliability for a temperature of the lower section of the tongue is higher than reliability for a temperature of the armpit, the reliability for the temperature of the arm pit is higher than reliability for a temperature of the ear drum, and the reliability for the temperature of the ear drum is higher than reliability for a temperature of a surface of the forehead.

As described above, reliability for a measured body temperature varies in accordance with a type of a thermometer and a measured portion. From these perspectives, in the second embodiment, the body temperature information is displayed in a distinctive display manner in accordance with the measurement device information in addition to the input mode, the measurement device information including at least one of information indicating whether a body temperature is measured in the predictive measurement manner or the actual measurement manner, information indicating whether the body temperature is measured in the contact manner or the contactless manner, and measured portion information indicating a body portion at which the body temperature has been measured.

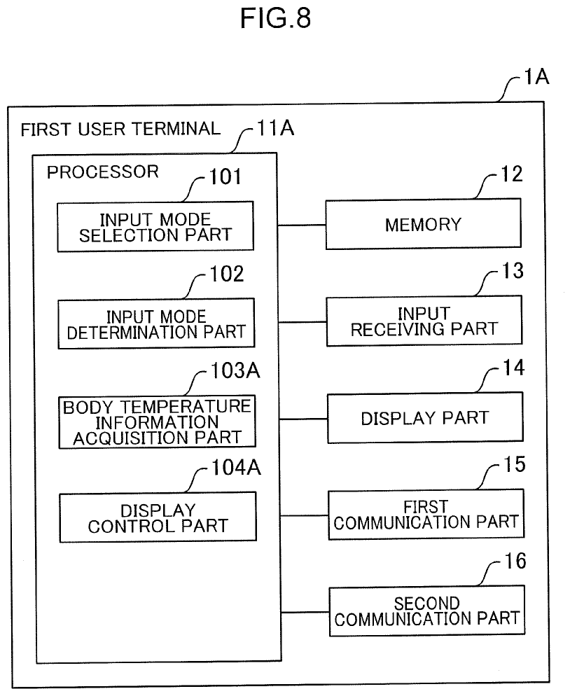

FIG. 8 is a diagram showing an example of a configuration of the first user terminal lA in the second embodiment of the disclosure.

The first user terminal 1A shown in FIG. 8 includes a processor 11A, a memory 12, an input receiving part 13, a display part 14, a first communication part 15, and a second communication part 16. The first user terminal 1A serves as an example of each of the first terminal and another terminal.

The processor 11A realizes an input mode selection part 101, an input mode determination part 102, a body temperature acquisition part 103A, and a display control part 104A. In the second embodiment, elements which are the same as those in the first embodiment are given the same reference signs and numerals, and thus explanation therefor will be omitted.

The body temperature acquisition part 103A acquires body temperature information in an input mode selected by the input mode selection part 101.

When the input mode selection part 101 selects a communication input mode, the body temperature acquisition part 103A acquires measurement device information together with the body temperature information from a thermometer 4 through communication therewith via the first communication part 15. The measurement device information includes at least one of information indicating whether a body temperature is measured in a predictive measurement manner or an actual measurement manner, information indicating whether the body temperature is measured in a contact manner or a contactless manner, and measured portion information indicating a body portion at which the body temperature has been measured. The thermometer 4 transmits the measurement device information together with the body temperature information to the first user terminal 1A. The first communication part 15 establishes the communication with the thermometer 4, and receives the body temperature information and the measurement device information from the thermometer 4.

The display control part 104A causes the display part 14 to display the body temperature information in a distinctive display manner in accordance with the input mode selected by the input mode selection part 101 and the measurement device information acquired by the body temperature acquisition part 103A. The display control part 104A executes displaying of a sign having a distinctive shape in accordance with the input mode in the vicinity of the body temperature information. The display control part 104A further executes displaying of a distinctive label in accordance with the measurement device information in the vicinity of the body temperature information. The label includes at least one of measurement of the body temperature in either the predictive manner or the actual measurement manner, measurement of the body temperature in either the contact manner or the contactless manner, and a body portion at which the body temperature has been measured. The display control part 104A may execute displaying of a sign having a distinctive color in accordance with the input mode in the vicinity of the body temperature information, may execute displaying of the body temperature information in a distinctive color in accordance with the input mode, or may execute displaying a name of the input mode in the vicinity of the body temperature information.

When the input mode is determined as the communication input mode, the display control part 104A causes the display part 14 to display the body temperature information in a third display manner, which is different from a first display manner and a second display manner, in accordance with the communication input mode and the measurement device information. When the input mode is determined as the manual input mode, the display control part 104A causes the display part 14 to display the body temperature information in the second display manner in accordance with the manual input mode.

The second communication part 16 transmits the measurement device information acquired by the body temperature acquisition part 103A together with the body temperature information acquired by the body temperature acquisition part 103A and the input mode identification information indicating the input mode selected by the input mode selection part 101. The second communication part 16 transmits the body temperature information, the input mode identification information, and the measurement device information to the body temperature management server 3.

The body temperature management server 3 receives the body temperature information, the input mode identification, and the measurement device information transmitted from the first user terminal 1A. The body temperature management server 3 transmits the received body temperature information about the first user, input mode identification information, and measurement device information to the second user terminal 2A.

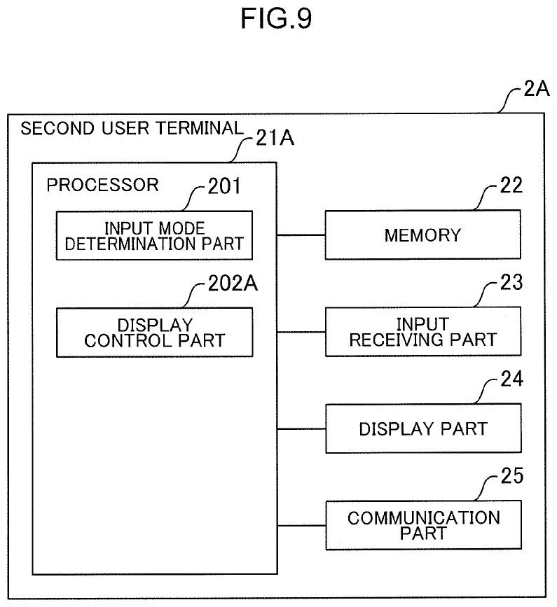

FIG. 9 is a diagram showing an example of a configuration of a second user terminal 2A in the second embodiment of the disclosure.

The second user terminal 2A shown in FIG. 9 includes a processor 21A, a memory 22, an input receiving part 23, a display part 24, and a communication part 25. The second user terminal 2A serves as an example of the second terminal and the terminal.

The processor 21A realizes an input mode determination part 201, and a display control part 202A. In the second embodiment, elements which are the same as those in the first embodiment are given the same reference signs and numerals, and thus explanation therefor will be omitted.

The communication part 25 receives measurement device information together with body temperature information and input mode identification information. The communication part 25 receives the body temperature information, the input mode identification information, and the measurement device information transmitted from the body temperature management server 3.

The display control part 202A causes the display part 24 to display the body temperature information in a distinctive display manner in accordance with the input mode identification information and the measurement device information. The display control part 202A executes displaying of a sign having a distinctive shape in accordance with the input mode identification information in the vicinity of the body temperature information. The display control part 202A further executes displaying of a distinctive label in accordance with the measurement device information in the vicinity of the body temperature information. The label includes at least one of measurement of the body temperature in either the predictive manner or the actual measurement manner, measurement of the body temperature in either the contact manner or the contactless manner, and a body portion at which the body temperature has been measured.

The display control part 202A may execute displaying of a sign having a distinctive color in accordance with the input mode identification information in the vicinity of the body temperature information, may execute displaying of the body temperature information in a distinctive color in accordance with the input mode identification information, or may execute displaying of a name of the input mode indicated by the input mode identification information in the vicinity of the body temperature information.

When the input mode is determined as the communication input mode, the display control part 202A causes the display part 24 to display the body temperature information in a third display manner, which is different from a first display manner and a second display manner, in accordance with the communication input mode and the measurement device information. When the input mode is determined as the manual input mode, the display control part 202A causes the display part 24 to display the body temperature information in the second display manner, which is different from the first display manner, in accordance with the manual input mode.

Next, acquisition of body temperature information by the first user terminal 1A in the second embodiment of the disclosure will be described.

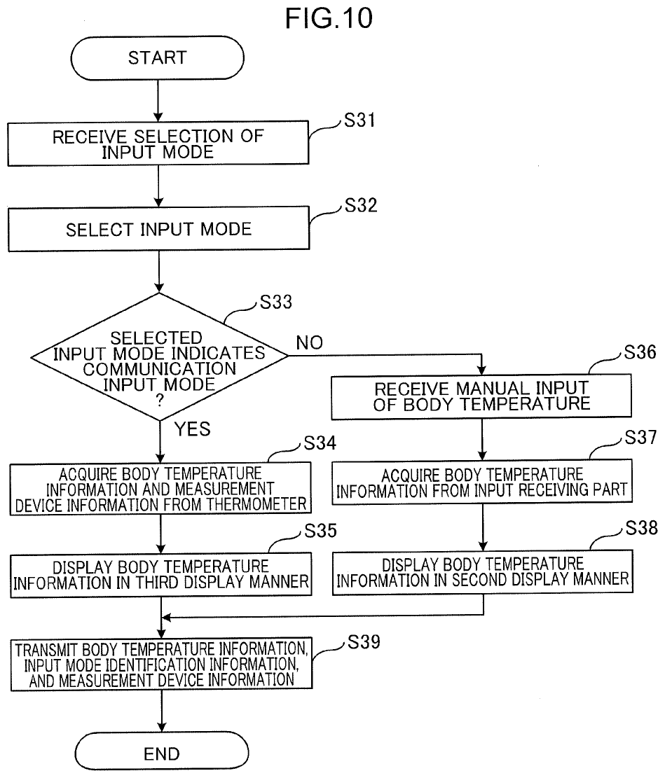

FIG. 10 is a flowchart explaining the acquisition of body temperature information by the first user terminal 1A in the second embodiment of the disclosure.

Steps S31 to S33 are the same as steps S1 to S3 in FIG. 4, and thus the descriptions therefor are omitted.

When a selected input mode is determined as the communication input mode (YES in step S33), the body temperature acquisition part 103A acquires, in step S34, body temperature information and measurement device information from the thermometer 4 through communication therewith via the first communication part 15. At this time, the first communication part 15 establishes a communication with the thermometer 4 and receives the body temperature information and the measurement device information from the thermometer 4 through a near field communication.

Then, in step S35, the display control part 104A causes the display part 14 to display the body temperature information in the third display manner in accordance with the communication input mode and the measurement device information. The display part 14 displays the body temperature information in the third display manner in response to an instruction from the display control part 104A.

By contrast, when the selected input mode is not determined as the communication input mode, that is, when the selected input mode is determined as the manual input mode (NO in step S33), the input receiving part 13 receives, in step S36, a manual input of a body temperature by the first user.

Steps S36 to S38 are the same as steps S6 to S8 in FIG. 4, and thus the descriptions therefor are omitted.

Subsequently, in step S39, the second communication part 16 transmits the body temperature information acquired by the body temperature acquisition part 103A, the input mode identification information indicating the input mode selected by the input mode selection part 101, and the measurement device information acquired by the body temperature acquisition part 103A.

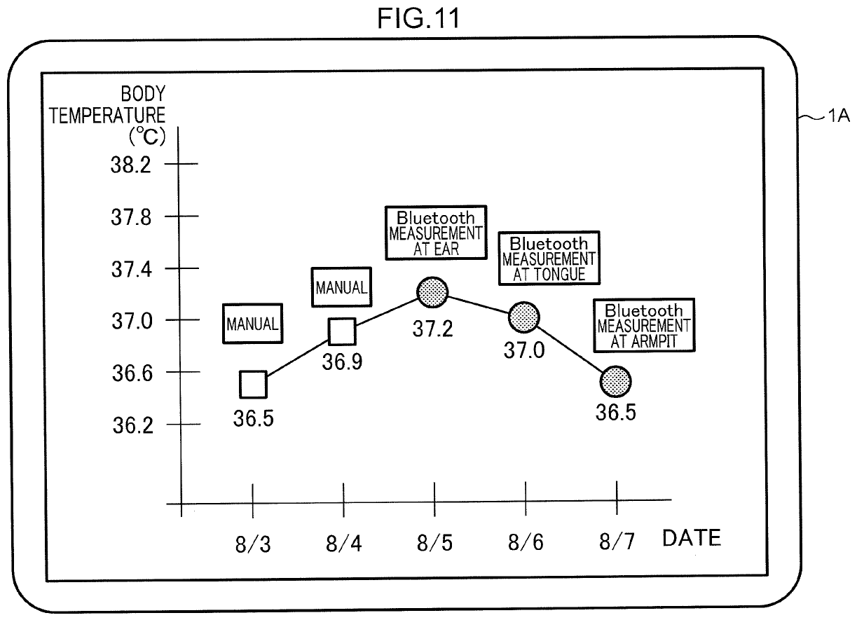

FIG. 11 shows an example of a body temperature presentation screen image displayed on the first user terminal 1A in the second embodiment.

As shown in FIG. 11, the display control part 104A executes the displaying of the body temperature information in the third display manner in accordance with the communication input mode and the measurement device information when the body temperature information is acquired in the communication input mode, and executes the displaying of the body temperature information in the second display manner in accordance with the manual input mode when the body temperature information is acquired in the manual input mode. The body temperature presentation screen image in FIG. 11 shows a history of body temperature information in a period from August 3^{rd} to August 7^{th} on a graph having a horizontal axis denoting a date and a vertical axis denoting a body temperature. The body temperature information on August 3^{rd} and August 4^{th} is acquired in the manual input mode, and the body temperature information in a period from August 5^{th} to August 7^{th} is acquired in the communication input mode.

When the body temperature information is acquired in the communication input mode, the display control part 104A executes displaying of a circular sign at a coordinate position corresponding to a date and a body temperature on the graph. The display control part 104A further executes the displaying of the circular sign in a red color. Besides, the display control part 104A executes displaying of a body temperature in a red color under the circular sign. Moreover, the display control part 104A executes displaying of a label indicating acquisition in the communication input mode above the circular sign. Examples of the label include the word “Bluetooth”. Moreover, the display control part 104A executes the displaying of the label indicating the measurement device information above the circular sign. Examples of the label include the terms “measurement at ear”, “measurement at tongue”, and “measurement at armpit”. The measurement device information in FIG. 11 shows each body portion at which the body temperature of the first user has been measured.

The second display manner for the body temperature information acquired in the manual input mode is the same as the manner described in the first embodiment, and thus the descriptions therefor are omitted.

Here, each of the shape and the color of each sign is a mere example, and another shape and another color may be adopted. Also, each color for each body temperature to be displayed under the relevant sign is a mere example, and another color may be adopted. Besides, the word or term of the label above the sign shows a mere example, and another word or term may be adopted. The display control part 104A may change only the shape of the sign indicating the body temperature for each of the communication input mode and the manual input mode. The display control part 104A may change only the color of the sign indicating the body temperature for each of the communication input mode and the manual input mode. The display control part 104A may avoid the displaying of the body temperature under the sign and avoid the displaying of the label above the sign.

The display control part 104A may execute displaying of a label showing measurement in either the predictive measurement manner or the actual measurement manner together with the body temperature information, or may execute displaying of a label showing measurement in either the contact manner or the contactless manner together with the body temperature information. The display control part 104A may execute displaying of a sign having a distinctive shape or distinctive color in accordance with the measurement in either the predictive measurement manner or the actual measurement manner, or may execute displaying of a sign having a distinctive shape or distinctive color in accordance with the measurement in either the contact manner or the contactless manner.

Furthermore, although the display control part 104A executes the displaying of the history of body temperature information on the graph, this disclosure is not particularly limited thereto, and the display control part may execute displaying of only body temperature information measured on an exact date.

Although the input receiving part 13 receives only a manual input of a body temperature by the first user when the manual input mode is selected in the second embodiment, the disclosure is not particularly limited thereto, and the input receiving part may receive a manual input of the body temperature and measurement device information by the first user. Specifically, the first user may manually input at least one of information indicating whether a body temperature is measured in the predictive measurement manner or the actual measurement manner, information indicating whether the body temperature is measured in the contact manner or the contactless manner, and measured portion information indicating a body portion at which the body temperature has been measured. When the input mode is determined as the manual input mode, the display control part 104A may cause the display part 14 to display the body temperature information in a fourth display manner in accordance with the manual input mode and the measurement device information.

As described heretofore, the body temperature information measured in the predictive measurement manner is displayed distinctively from the body temperature information measured in the actual measurement manner, and therefore, reliability for the body temperature information is presentable to the first user. Moreover, the body temperature information measured in the contact manner is displayed distinctively from the body temperature information measured in the contactless manner, and therefore, reliability for the body temperature information is presentable to the first user. In addition, the body temperature information is displayed in a distinctive display manner in accordance with a body portion at which the body temperature has been measured, and thus, reliability for the body temperature information is presentable to the first user.

Next, presentation of body temperature information by the second user terminal 2A in the second embodiment of the disclosure will be described.

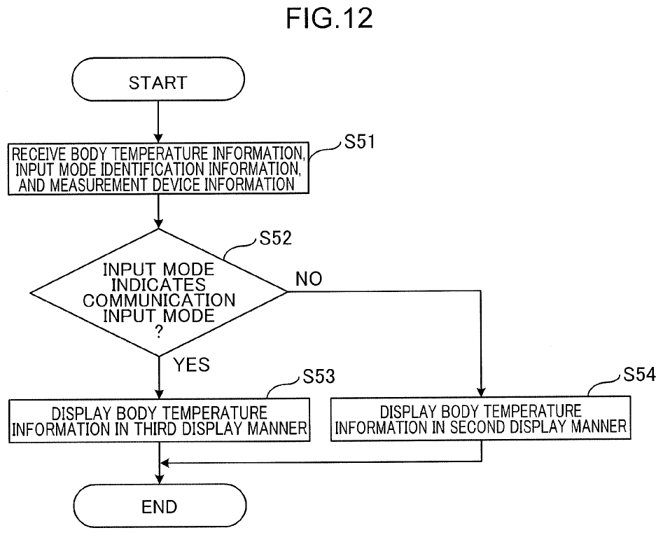

FIG. 12 is a flowchart explaining the presentation of body temperature information by the second user terminal 2A in the second embodiment of the disclosure.

First, in step S51, the communication part 25 receives body temperature information, input mode identification information, and measurement device information.

Next, in step S25, the input mode determination part 201 determines whether an input mode indicated by the input mode identification information received by the communication part 25 indicates the communication input mode.

Here, when the input mode is determined as the communication input mode (YES in step S52), the display control part 202A causes, in step S53, the display part 24 to display the body temperature information in the third display manner in accordance with the communication input mode and the measurement device information. The display part 24 displays the body temperature information in the third display manner in response to an instruction from the display control part 202A.

By contrast, when the input mode is not determined as the communication input mode, that is, when the input mode is determined as the manual input mode (NO in step S52), the display control part 202A causes, in step S54, the display part 24 to display the body temperature information in the second display manner in accordance with the manual input mode. The display part 24 displays the body temperature information in the second display manner in response to an instruction from the display control part 202A.

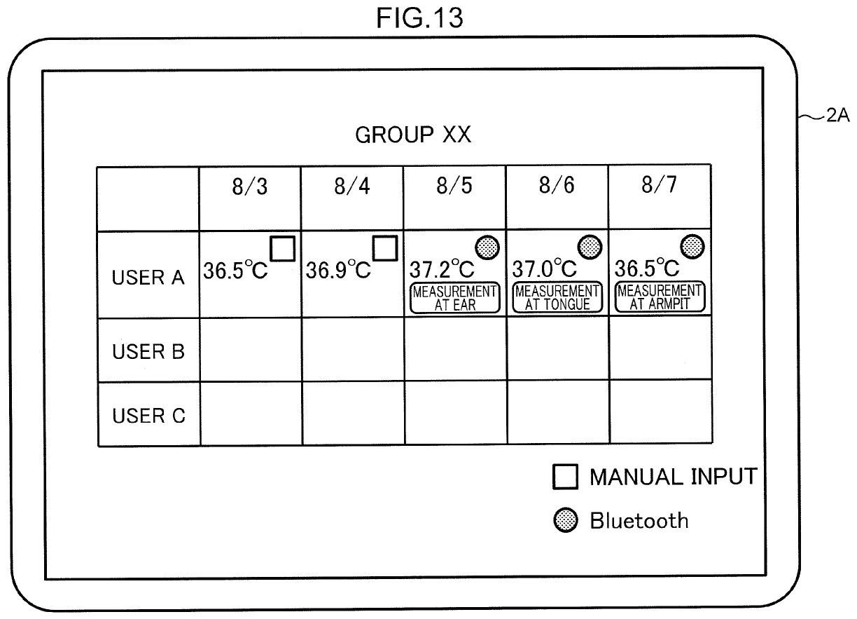

FIG. 13 shows an example of a body temperature presentation screen image displayed on the second user terminal 2A in the second embodiment.

As shown in FIG. 13, the display control part 202A executes the displaying of the body temperature information in the third display manner in accordance with the communication input mode and the measurement device information when the body temperature information is acquired in the communication input mode, and executes the displaying of the body temperature information in the second display manner in accordance with the manual input mode when the body temperature information is acquired in the manual input mode. The body temperature presentation screen image in FIG. 13 shows a history of body temperature information about each first user in a period from August 3^{rd} to August 7^{th} on a table associating a name of a member belonging to a group managed by the second user with a relevant date. The body temperature information about a user A on August 3^{rd} and August 4^{th} is acquired in the manual input mode, and the body temperature information about the user A in a period from August 5^{th} to August 7^{th} is acquired in the communication input mode.

When the body temperature information is acquired in the communication input mode, the display control part 202A executes displaying of a body temperature at a position corresponding to a user name and a date on the table, and executes displaying of a circular sign at an upper right position of the body temperature. The display control part 202A further executes the displaying of the circular sign in a red color. Besides, the display control part 202A executes the displaying of the body temperature in a red color. Moreover, the display control part 202A executes displaying of a label indicating the measurement device information below the body temperature. Examples of the label include the terms “measurement at ear”, “measurement at tongue”, and “measurement at armpit”. The measurement device information in FIG. 13 shows each body portion at which the body temperature of the first user has been measured.

The second display manner for the body temperature information acquired in the manual input mode is the same as the manner described in the first embodiment, and thus the descriptions therefor are omitted.

Here, each of the shape and the color of each sign is a mere example, and another shape and another color may be adopted. Also, each color for each body temperature to be displayed is a mere example, and another color may be adopted. The display control part 202A may change only the shape of the sign indicating each of the communication input mode and the manual input mode. The display control part 202A may change only the color of the sign indicating each of the communication input mode and the manual input mode.

The display control part 202A may execute displaying of a label showing measurement in either the predictive measurement manner or the actual measurement manner together with the body temperature information, or may execute displaying of a label showing measurement in either the contact manner or the contactless manner together with the body temperature information. The display control part 202A may execute displaying of a sign having a distinctive shape or distinctive color in accordance with the measurement in either the predictive measurement manner or the actual measurement manner, or may execute displaying of a sign having a distinctive shape or distinctive color in accordance with the measurement in either the contact manner or the contactless manner.

Furthermore, although the display control part 202A executes the displaying of the history of body temperature information on the table, this disclosure is not particularly limited thereto, and the display control part may execute displaying of only body temperature information about the first user measured on an exact date. In addition, the display control part 202A may execute the displaying of the history of body temperature information about the first user on the graph shown in FIG. 11.

Although the display control part 202A causes the display part 24 to display the body temperature information in the second display manner in accordance with the manual input mode when the input mode is determined as the manual input mode in the second embodiment, the disclosure is not particularly limited thereto. When the first user terminal 1A receives a manual input of a body temperature and measurement device information by the first user, the display control part 202A may cause the display part 24 to display body temperature information in a fourth display manner in accordance with the manual input mode and the measurement device information.

As described heretofore, the body temperature information measured in the predictive measurement manner is displayed distinctively from the body temperature information measured in the actual measurement manner, and therefore, reliability for the body temperature information about the first user is presentable to the second user. Furthermore, the body temperature information measured in the contact manner is displayed distinctively from the body temperature information measured in the contactless manner, and therefore, the reliability for the body temperature information about the first user is presentable to the second user. In addition, the body temperature information is displayed in a distinctive display manner in accordance with a body portion at which the body temperature has been measured, and thus, reliability for the body temperature information about the first user is presentable to the second user.

### Third Embodiment

In the second embodiment, the first user terminal 1A acquires body temperature information and measurement device information from the thermometer 4. By contrast, in a third embodiment, a first user terminal 1B acquires a device ID identifying a thermometer 4 and body temperature information from the thermometer 4, and the first user terminal 1B and a body temperature management server 3 acquire measurement device information by using the device ID.

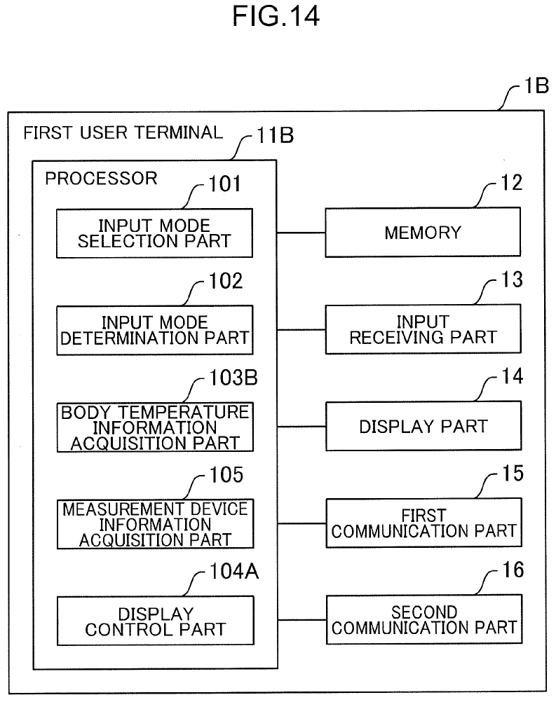

FIG. 14 is a diagram showing an example of a configuration of the first user terminal 1B in the third embodiment of the disclosure. A body temperature management system in the third embodiment includes the first user terminal 1B, a second user terminal 2A, and the body temperature management server 3.

The first user terminal 1B shown in FIG. 14 includes a processor 11B, a memory 12, an input receiving part 13, a display part 14, a first communication part 15, and a second communication part 16. The first user terminal 1B serves as an example of each of the first terminal and another terminal.

The processor 11B realizes an input mode selection part 101, an input mode determination part 102, a body temperature acquisition part 103B, a display control part 104A, and a measurement device information acquisition part 105. In the third embodiment, elements which are the same as those in the first embodiment and the second embodiment are given the same reference signs and numerals, and thus explanation therefor will be omitted.

The body temperature acquisition part 103B acquires body temperature information in an input mode selected by the input mode selection part 101.

When the input mode selection part 101 selects a communication input mode, the body temperature acquisition part 103B acquires a device ID (device identification information) together with the body temperature information from the thermometer 4 through communication therewith via the first communication part 15. The device ID represents information for identifying the thermometer 4 which measures a body temperature of a first user. The thermometer 4 transmits the device ID together with the body temperature information to the first user terminal 1B. The first communication part 15 establishes a communication with the thermometer 4 and receives the body temperature information and the device ID from the thermometer 4.

The measurement device information acquisition part 105 acquires measurement device information from the body temperature management server 3. The body temperature management server 3 stores the device ID and the measurement device information in association with each other. The second communication part 16 transmits the device ID acquired by the body temperature acquisition part 103B to the body temperature management server 3, and receives the measurement device information associated with the device ID from the body temperature management server 3. The measurement device information acquisition part 105 acquires, from the second communication part 16, the measurement device information received from the body temperature management server 3.

Here, the memory 12 may store a device ID and measurement device information in association with each other in advance. The measurement device information acquisition part 105 may acquire the measurement device information associated with the acquired device ID from the memory 12. The measurement device information acquisition part 105 may acquire the measurement device information from an external server which is different from the body temperature management server 3.

The display control part 104A causes the display part 14 to display the body temperature information in a distinctive display manner in accordance with the input mode selected by the input mode selection part 101 and the measurement device information acquired by the measurement device information acquisition part 105.

The second communication part 16 transmits the device ID acquired by the body temperature acquisition part 103B together with the body temperature information acquired by the body temperature acquisition part 103B and the input mode identification information indicating the input mode selected by the input mode selection part 101. The second communication part 16 transmits the body temperature information, the input mode identification information, and the device ID to the body temperature management server 3.

The body temperature management server 3 receives the body temperature information, the input mode identification information, and the device ID transmitted from the first user terminal 1B. The body temperature management server 3 specifies measurement device information associated with the received device ID in advance. A memory included in the body temperature management server 3 stores the device ID and the measurement device information in association with each other. A processor included in the body temperature management server 3 acquires, from the memory, the measurement device information associated with the received device ID. The body temperature management server 3 transmits the received body temperature information about the first user, input mode identification information, and measurement device information to the second user terminal 2A.

The second user terminal 2A in the third embodiment has the same configuration as the second user terminal 2A in the second embodiment.

A communication part 25 included in the second user terminal 2A receives measurement device information specified on the basis of a device ID together with body temperature information and input mode identification information. A display control part 202A causes a display part 24 to display the body temperature information in a distinctive display manner in accordance with the input mode identification information and the measurement device information.

Although the second user terminal 2A receives the body temperature information about the first user, the input mode identification information, and the measurement device information from the body temperature management server 3 in the third embodiment, the disclosure is not particularly limited thereto, and the second user terminal 2A may receive the body temperature information about the first user, the input mode identification information, and the device ID from the body temperature management server 3. In this case, a processor 21A included in the second user terminal 2A may include a measurement device information acquisition part. The measurement device information acquisition part of the processor 21A may acquire the measurement device information from the body temperature management server 3. The communication part 25 may transmit the received device ID to the body temperature management server 3, and receive measurement device information associated with the device ID from the body temperature management server 3. The measurement device information acquisition part may acquire, from the communication part 25, the measurement device information received from the body temperature management server 3.

Next, acquisition of body temperature information by the first user terminal 1B in the third embodiment of the disclosure will be described.

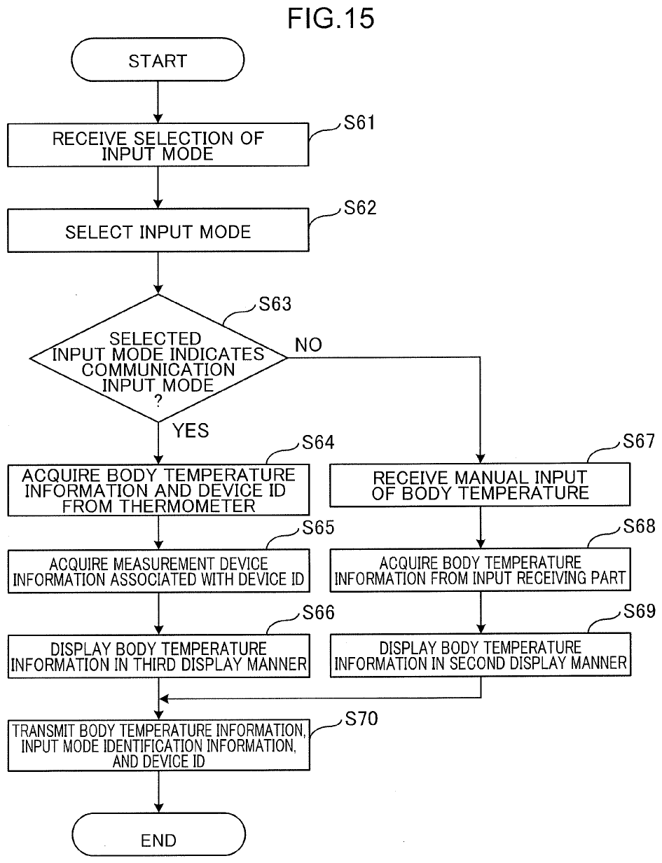

FIG. 15 is a flowchart explaining the acquisition of body temperature information by the first user terminal 1B in the third embodiment of the disclosure.

Steps S61 to S63 are the same as steps S1 to S3 in FIG. 4, and thus the descriptions therefor are omitted.

When a selected input mode is determined as the communication input mode (YES in step S63), the body temperature acquisition part 103B acquires, in step S64, body temperature information and a device ID from the thermometer 4 through communication therewith via the first communication part 15. At this time, the first communication part 15 establishes a communication with the thermometer 4 and receives the body temperature information and the device ID from the thermometer 4 through a near field communication.

Subsequently, in step S65, the measurement device information acquisition part 105 acquires, from the body temperature management server 3, measurement device information associated with the device ID acquired by the body temperature acquisition part 103B. At this time, the second communication part 16 transmits the device ID acquired by the body temperature acquisition part 103B to the body temperature management server 3, and receives measurement device information associated with the device ID from the body temperature management server 3. The measurement device information acquisition part 105 acquires the measurement device information from the second communication part 16.

Then, in step S66, the display control part 104A causes the display part 14 to display the body temperature information in a third display manner in accordance with the communication input mode and the measurement device information. The display part 14 displays the body temperature information in the third display manner in response to an instruction from the display control part 104A.

By contrast, when the selected input mode is not determined as the communication input mode, that is, when the selected input mode is determined as the manual input mode (NO in step S63), the input receiving part 13 receives, in step S67, a manual input of a body temperature by the first user.

Steps S67 to S69 are the same as steps S6 to S8 in FIG. 4, and thus the descriptions therefor are omitted.

Subsequently, in step S70, the second communication part 16 transmits the body temperature information acquired by the body temperature acquisition part 103B, the input mode identification information indicating the input mode selected by the input mode selection part 101, and the device ID acquired by the body temperature acquisition part 103B.

It is noted that presentation of body temperature information by the second user terminal 2A in the third embodiment is the same as the presentation of body temperature information by the second user terminal 2A in the second embodiment.

Besides, in the third embodiment, the second communication part 16 may transmit the body temperature information acquired by the body temperature acquisition part 103B, the input mode identification information indicating the input mode selected by the input mode selection part 101, and the measurement device information acquired by the measurement device information acquisition part 105.

Although the input receiving part 13 of the first user terminal 1B receives only a manual input of a body temperature by the first user when the manual input mode is selected in the third embodiment, the disclosure is not particularly limited thereto, and the input receiving part 13 may receive a manual input of the body temperature and the device ID by the first user. In other words, the first user may manually input the device ID of the thermometer 4. The measurement device information acquisition part 105 may acquire, from the body temperature management server 3, the measurement device information associated with the device ID received by the input receiving part 13. When the input mode is determined as the manual input mode, the display control part 104A may cause the display part 14 to display the body temperature information in a fourth display manner in accordance with the manual input mode and the measurement device information.

Although the display control part 202A of the second user terminal 2A causes the display part 24 to display the body temperature information in the second display manner in accordance with the manual input mode when the input mode is determined as the manual input mode in the third embodiment, the disclosure is not particularly limited thereto. When the first user terminal 1B receives a manual input of a body temperature and a device ID by the first user, the display control part 202A may cause the display part 24 to display body temperature information in a fourth display manner in accordance with the manual input mode and the measurement device information.

The device ID having a data amount smaller than that of the measurement device information is transmitted from the thermometer 4 to the first user terminal 1B as described above, and therefore, reduction in the data amount of communication between the thermometer 4 and the first user terminal 1B is achieved and a shorter time for the communication is attained. Moreover, the device ID is transmitted from the first user terminal 1B to the body temperature management server 3, and therefore, reduction in the data amount of communication between the first user terminal 1B and the body temperature management server 3 is achieved and a shorter time for the communication is attained.

### Fourth Embodiment

Providing a privilege to a user having continuously measured a body temperature thereof in an input mode having high reliability leads to achievement in encouraging the user to continuously measure the body temperature thereof in the input mode having the high reliability. In this respect, a good health condition certificate proving that the user has a good health condition is issued as the privilege to the user having continuously measured a body temperature thereof in the input mode having the high reliability.

In a fourth embodiment, a first user terminal further generates a good health condition certificate proving that a first user has a good health condition when a selected input mode continuously indicates a communication input mode throughout a predetermined period and acquired body temperature information continuously falls below a predetermined value throughout a predetermined period.

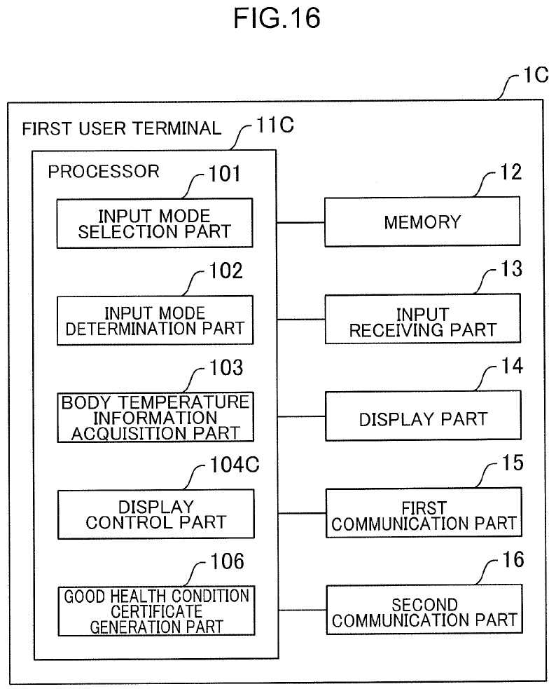

FIG. 16 is a diagram showing an example of a configuration of a first user terminal 1C in the fourth embodiment of the disclosure. A body temperature management system in the fourth embodiment includes the first user terminal 1C, a second user terminal 2, and a body temperature management server 3. The second user terminal 2 in the fourth embodiment has the same configuration as the second user terminal 2 in the first embodiment.

The first user terminal 1C shown in FIG. 16 includes a processor 11C, a memory 12, an input receiving part 13, a display part 14, a first communication part 15, and a second communication part 16. The first user terminal 1C serves as an example of each of the first terminal and another terminal.

The processor 11C realizes an input mode selection part 101, an input mode determination part 102, a body temperature acquisition part 103, a display control part 104C, and a good health condition certificate generation part 106. In the fourth embodiment, elements which are the same as those in the first embodiment are given the same reference signs and numerals, and thus explanation therefor will be omitted.

The good health condition certificate generation part 106 generates a good health condition certificate proving that the first user has a good health condition when an input mode selected by the input mode selection part 101 continuously indicates the communication input mode throughout a predetermined period and body temperature information acquired by the body temperature acquisition part 103 continuously falls below a predetermined value throughout a predetermined period.

The predetermined period includes, for example, seven days. The predetermined value is, for example, 37.0° C. defined as a determination criterion for having a fever. The memory 12 stores the predetermined period and the predetermined value in advance. The second communication part 16 may receive a predetermined period on request from the body temperature management server 3.

The memory 12 further stores a history of body temperature information about the first user, and a history of each input mode used in acquiring the body temperature information. The body temperature management server 3 may store the history of body temperature information and the history of each input mode.

The display control part 104C causes the display part 14 to display the body temperature information in a distinctive display manner in accordance with the input mode selected by the input mode selection part 101. The display control part 104C further causes the display part 14 to display the good health condition certificate generated by the good health condition certificate generation part 106.

Next, acquisition of body temperature information by the first user terminal 1C in the fourth embodiment of the disclosure will be described.

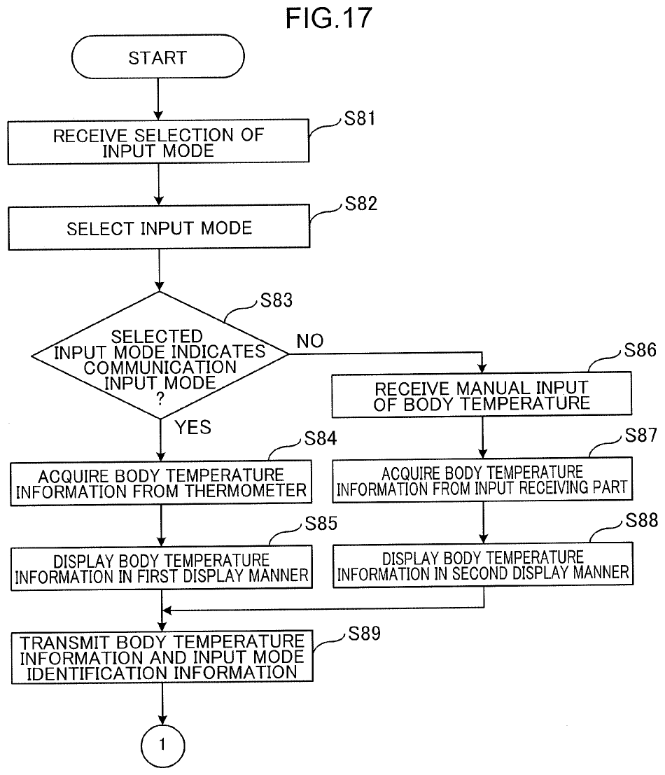

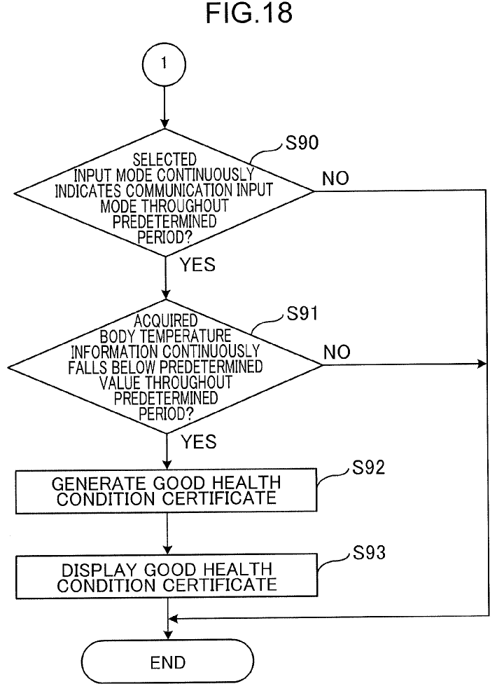

FIG. 17 is a first flowchart explaining the acquisition of body temperature information by the first user terminal 1C in the fourth embodiment of the disclosure. FIG. 18 is a second flowchart explaining the acquisition of body temperature information by the first user terminal 1C in the fourth embodiment of the disclosure.

Steps S81 to S89 are the same as steps S1 to S9 in FIG. 4, and thus the descriptions therefor are omitted.

Next, in step S90, the good health condition certificate generation part 106 determines whether an input mode selected by the input mode selection part 101 continuously indicates the communication input mode throughout a predetermined period.

Here, when the selected input mode is not determined to continuously indicate the communication input mode throughout the predetermined period, that is, when the predetermined period includes at least one day when a selected input mode indicates the manual input mode (NO in step S90), the flow finishes there.

By contrast, when the selected input mode is determined to continuously indicate the communication input mode throughout the predetermined period (YES in step S90), the good health condition certificate generation part 106 determines, in step S91, whether the body temperature information acquired by the body temperature acquisition part 103 continuously falls below a predetermined value throughout a predetermined period.

Here, when the acquired body temperature information is not determined to continuously fall below the predetermined value throughout the predetermined period, that is, the predetermined period includes at least one day when acquired body temperature information indicates the predetermined value or higher (NO in step S91), the flow finishes there.

Contrarily, when the acquired body temperature information is determined to continuously fall below the predetermined value throughout the predetermined period (YES in step S91), the good health condition certificate generation part 106 generates, in step S92, a good health condition certificate proving that the first user has a good health condition.

Subsequently, in step S93, the display control part 104C causes the display part 14 to display the good health condition certificate generated by the good health condition certificate generation part 106. The display part 14 displays the good health condition certificate in response to an instruction from the display control part 104C.

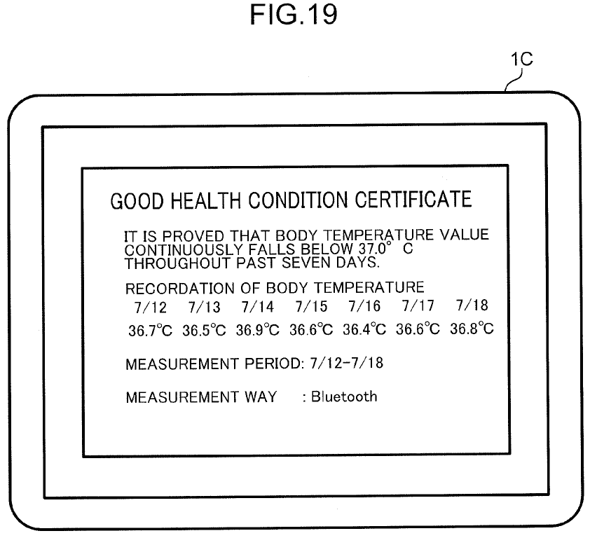

FIG. 19 shows an example of a good health condition certificate presentation screen image displayed on the first user terminal 1C in the fourth embodiment.

As shown in FIG. 19, the display control part 104C executes displaying of the good health condition certificate when a selected input mode continuously indicates the communication input mode throughout seven days and acquired body temperature information continuously falls below 37.0° C. throughout the seven days. The displayed good health condition certificate presentation screen image in FIG. 19 includes: a sentence confirming a body temperature falling below 37.0° C. throughout past seven days; values of the body temperature in the period of past seven days; a measurement period; and a measurement way. The measurement way shows acquisition of the body temperature information through the Bluetooth.

In the fourth embodiment, when the body temperature acquisition part 103 can acquire measurement device information, the good health condition certificate generation part 106 may generate a good health condition certificate including at least one of measurement of a body temperature in either a predictive measurement manner or an actual measurement manner, measurement of the body temperature in either a contact manner or a contactless manner, and a body portion at which the body temperature has been measured.

As described heretofore, a good health condition certificate proving that the first user has a good health condition is generated and displayed when the selected input mode continuously indicates the communication input mode throughout the predetermined period and the acquired body temperature information continuously falls below the predetermined value throughout the predetermined period, and therefore, the continuous good health condition of the first user throughout the predetermined period is presentable.

Although the good health condition certificate generation part 106 generates a good health condition certificate when an input mode selected by the input mode selection part 101 continuously indicates the communication input mode throughout a predetermined period and body temperature information acquired by the body temperature acquisition part 103 continuously falls below a predetermined value throughout a predetermined period in the fourth embodiment, the disclosure is not particularly limited thereto. The good health condition certificate generation part 106 may generate a good health condition certificate when an input mode selected by the input mode selection part 101 indicates the communication input mode and body temperature information acquired by the body temperature acquisition part 103 falls below the predetermined value.

Besides, the good health condition certificate generation part 106 may only determine whether acquired body temperature information continuously falls below a predetermined value throughout a predetermined period without determining whether a selected input mode continuously indicates the communication input mode throughout a predetermined period. In this case, the good health condition certificate generation part 106 may generate the good health condition certificate when the acquired body temperature information continuously falls below the predetermined value throughout the predetermined period.

Furthermore, although the processor 11C of the first user terminal 1C realizes the good health condition certificate generation part 106 in the fourth embodiment, the disclosure is not limited thereto, and a processor included in the body temperature management server 3 may realize the good health condition certificate generation part 106. Specifically, the body temperature management server 3 may generate a good health condition certificate when a selected input mode continuously indicates the communication input mode throughout the predetermined period and acquired body temperature information continuously falls below the predetermined value throughout the predetermined period. The body temperature management server 3 may transmit the generated good health condition certificate to the first user terminal 1C.

The input receiving part 13 may receive an input of health condition information by the first user when a body temperature is measured. The good health condition certificate generation part 106 may acquire, from the input receiving part 13, health condition information indicating a health condition of the first user. The health condition information indicates the health condition of the first user, such as, coughing, feeling of fatigue, or goodness. The input receiving part 13 receives a selection of at least one item by the first user from among a plurality of items each showing a health condition. The items include, for example, goodness, coughing, feeling of fatigue, a sore throat, diarrhea, a headache, and a taste disorder. The good health condition certificate generation part 106 may generate a good health condition certificate including body temperature information, and health condition information in acquisition of the body temperature information.

In addition, in a modification of the fourth embodiment, a good health condition certificate generation part 106 may generate a good health condition certificate for a first user on the basis of body temperature information and health condition information about another user. The good health condition certificate generation part 106 may generate a good health condition certificate when body temperature information about another user who works in the same space as the first user continuously falls below a predetermined value throughout a predetermined period and the health condition information about another user continuously indicates goodness throughout a predetermined period.

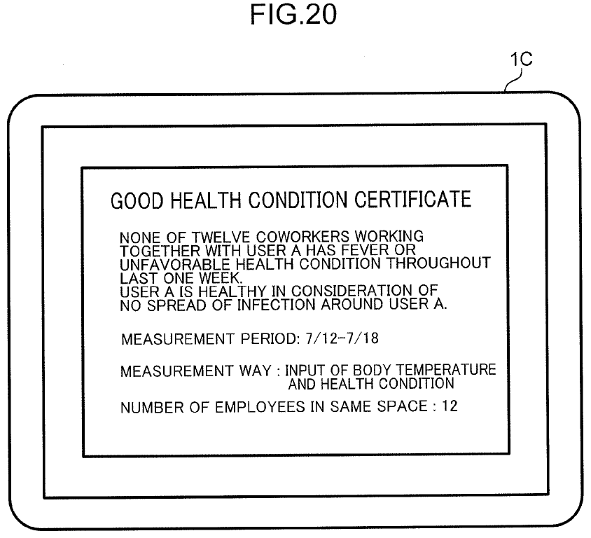

FIG. 20 shows an example of a good health condition certificate presentation screen image displayed on a first user terminal 1C in the modification of the fourth embodiment.

As shown in FIG. 20, a display control part 104C displays a good health condition certificate when body temperature information about another user working together with the first user in the same space falls below 37.0° C. throughout seven days and health condition information about another user continuously indicates goodness throughout the seven days. The displayed good health condition certificate presentation screen image in FIG. 20 includes: a sentence confirming no person having a fever and no person having an unfavorable health condition among other users or workers working together with a user A (first user) in the same space throughout the past seven days; a measurement period; a measurement way; and the number of workers in the same space. The measurement way shows acquisition of the body temperature information and the health condition information.

The good health condition certificate generation part 106 may generate a first reliability level-good health condition certificate showing a highest reliability level when body temperature information about all the users among a plurality of users in a group to which the first user belongs continuously falls below a predetermined value throughout a predetermined period. The good health condition certificate generation part 106 may generate a second reliability level-good health condition certificate showing a second highest reliability level when body temperature information about 80% of the users in the group to which the first user belongs continuously falls below the predetermined value throughout the predetermined period. The good health condition certificate generation part 106 may generate a third reliability level-good health condition certificate showing a third highest reliability level when body temperature information about 60% of the users in the group to which the first user belongs continuously falls below the predetermined value throughout the predetermined period.

The good health condition certificate generation part 106 may generate the first reliability level-good health condition certificate showing the highest reliability level when an input mode by at least one of the other users in the group to which the first user belongs continuously indicates the communication input mode throughout a predetermined period and acquired body temperature information about the first user continuously falls below a predetermined value throughout a predetermined period.

The good health condition certificate generation part 106 may generate the second reliability level-good health condition certificate showing the second highest reliability level when an input mode by at least one of the other users in the group to which the first user belongs continuously indicates the manual input mode throughout a predetermined period and acquired body temperature information about the first user continuously falls below a predetermined value throughout a predetermined period.

The good health condition certificate generation part 106 may generate the first reliability level-good health condition certificate showing the highest reliability level when a selected input mode continuously indicates the communication input mode throughout a predetermined period and acquired body temperature information continuously falls below a predetermined value throughout a predetermined period. The good health condition certificate generation part 106 may generate the second reliability level-good health condition certificate showing the second highest reliability level when a selected input mode continuously indicates the manual input mode throughout a predetermined period and acquired body temperature information continuously falls below a predetermined value throughout a predetermined period. The good health condition certificate generation part 106 may generate the third reliability level-good health condition certificate showing the third highest reliability level when a selected input mode continuously indicates the manual input mode throughout a predetermined period, acquired body temperature information continuously falls below a predetermined value throughout a predetermined period, and acquired body temperature information continuously indicates a value equal to a default value throughout a predetermined period.

Specifically, the first user manually inputs a body temperature by increasing or decreasing the default value (e.g., 36.5° C.) for the body temperature displayed on a body temperature input screen image. When the input body temperature continuously indicates a value equal to the default value throughout a predetermined period, a possibility of input of wrong body temperature information by the first user increases. Hence, the good health condition certificate generation part 106 may generate the third reliability level-good health condition certificate showing the third highest reliability level when a selected input mode continuously indicates the manual input mode throughout a predetermined period, the acquired body temperature information continuously falls below a predetermined value throughout a predetermined period, and the acquired body temperature information continuously indicates a value equal to a default value throughout the predetermined period.

### Fifth Embodiment

Providing a privilege to a user having continuously measured a body temperature thereof in an input mode having high reliability leads to achievement in encouraging the user to continuously measure the body temperature thereof in the input mode having the high reliability. For instance, when the body temperature of 37.5° C. or higher continues for four or more days, a infection from a specific virus, such as novel coronavirus, is suspected. In this regard, a preferential virus test certificate proving that a user is preferentially prompted to receive a virus test is issued as a privilege to the user having continuously measured a body temperature thereof in an input mode having high reliability.

In a fifth embodiment, a first user terminal further generates a preferential virus test certificate proving that a first user is preferentially prompted to receive a virus test when a selected input mode continuously indicates a communication input mode throughout a predetermined period and acquired body temperature information continuously indicates a predetermined value or higher throughout a predetermined period.

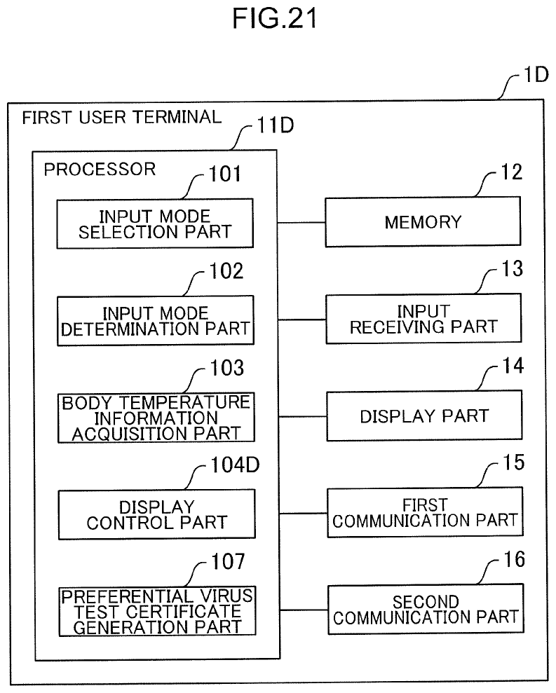

FIG. 21 is a diagram showing an example of a configuration of a first user terminal 1D in the fifth embodiment of the disclosure. A body temperature management system in the fifth embodiment includes the first user terminal 1D, a second user terminal 2, and a body temperature management server 3. The second user terminal 2 in the fifth embodiment has the same configuration as the second user terminal 2 in the first embodiment.

The first user terminal 1D shown in FIG. 21 includes a processor 11D, a memory 12, an input receiving part 13, a display part 14, a first communication part 15, and a second communication part 16. The first user terminal 1D serves as an example of each of the first terminal and another terminal.

The processor 11D realizes an input mode selection part 101, an input mode determination part 102, a body temperature acquisition part 103, a display control part 104D, and a preferential virus test certificate generation part 107. In the fifth embodiment, elements which are the same as those in the first embodiment are given the same reference signs and numerals, and thus explanation therefor will be omitted.

The preferential virus test certificate generation part 107 generates a preferential virus test certificate proving that the first user is preferentially prompted to receive a virus test when the input mode selected by the input mode selection part 101 continuously indicates the communication input mode throughout a predetermined period and body temperature information acquired by the body temperature acquisition part 103 continuously indicates a predetermined value or higher throughout a predetermined period.

The predetermined period includes, for example, four days. The predetermined value is, for example, 37.5° C. defined as a determination criterion for a virus infection. The memory 12 stores the predetermined period and the predetermined value in advance. The second communication part 16 may receive a predetermined period on request from the body temperature management server 3. The virus test, is for example, a PCR (polymerase chain reaction) test, an antigen test, or an antibody test.

The memory 12 further stores a history of body temperature information about the first user, and a history of each input mode used in acquiring the body temperature information. The body temperature management server 3 may store the history of body temperature information and the history of each input mode.

A predetermined period for determining whether a selected input mode continuously indicates the communication input mode may be the same as or different from a predetermined period for determining whether acquired body temperature information continuously indicates a predetermined value or higher. The predetermined period for determining whether the selected input mode continuously indicates the communication input mode may include, for example, seven days, and the predetermined period for determining whether acquired body temperature information continuously indicates the predetermined value or higher may include, for example, four days.

The display control part 104D causes the display part 14 to display the body temperature information in a distinctive display manner in accordance with the input mode selected by the input mode selection part 101. The display control part 104D causes the display part 14 to display the preferential virus test certificate generated by the preferential virus test certificate generation part 107.

Next, acquisition of body temperature information by the first user terminal 1D in the fifth embodiment of the disclosure will be described.

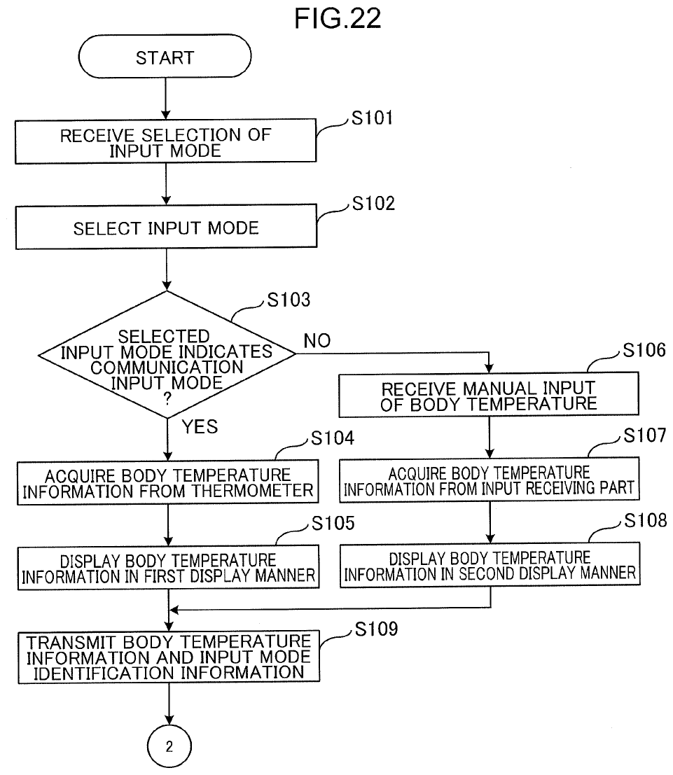

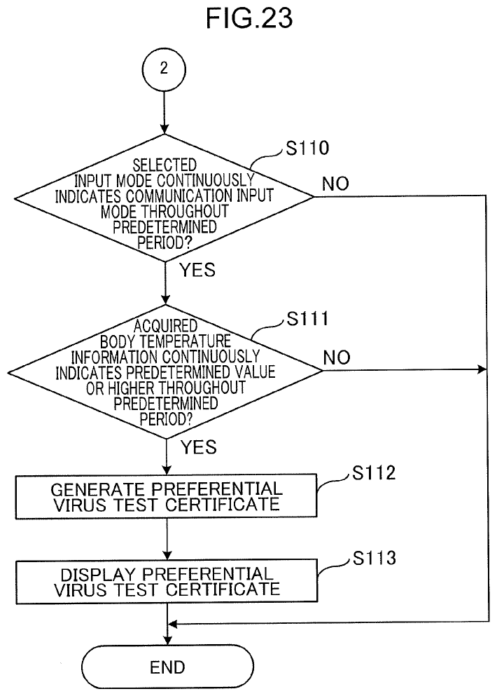

FIG. 22 is a first flowchart explaining the acquisition of body temperature information by the first user terminal 1D in the fifth embodiment of the disclosure. FIG. 23 is a second flowchart explaining the acquisition of body temperature information by the first user terminal 1D in the fifth embodiment of the disclosure.

Steps S101 to S109 are the same as steps S1 to S9 in FIG. 4, and thus the descriptions therefor are omitted.

Next, in step S110, the preferential virus test certificate generation part 107 determines whether an input mode selected by the input mode selection part 101 continuously indicates the communication input mode throughout a predetermined period.

Here, when the selected input mode is not determined to continuously indicate the communication input mode throughout the predetermined period, that is, when the predetermined period includes at least one day when a selected input mode indicates the manual input mode (NO in step S110), the flow finishes there.

By contrast, when the selected input mode is determined to continuously indicate the communication input mode throughout the predetermined period (YES in step S110), the preferential virus test certificate generation part 107 determines, in step S111, whether the body temperature information acquired by the body temperature acquisition part 103 continuously indicates a predetermined value or higher throughout a predetermined period.

Here, when the acquired body temperature information is not determined to continuously indicate the predetermined value or higher throughout the predetermined period, that is, the predetermined period includes at least one day when acquired body temperature information falls below the predetermined value (NO in step S111), the flow finishes there.

Contrarily, when the acquired body temperature information is determined to continuously indicate the predetermined value or higher throughout the predetermined period (YES in step S111), the preferential virus test certificate generation part 107 generates, in step S112, a preferential virus test certificate proving that the first user is preferentially prompted to receive a virus test.

Subsequently, in step S113, the display control part 104D causes the display part 14 to display the preferential virus test certificate generated by the preferential virus test certificate generation part 107. The display part 14 displays the preferential virus test certificate in response to an instruction from the display control part 104D.

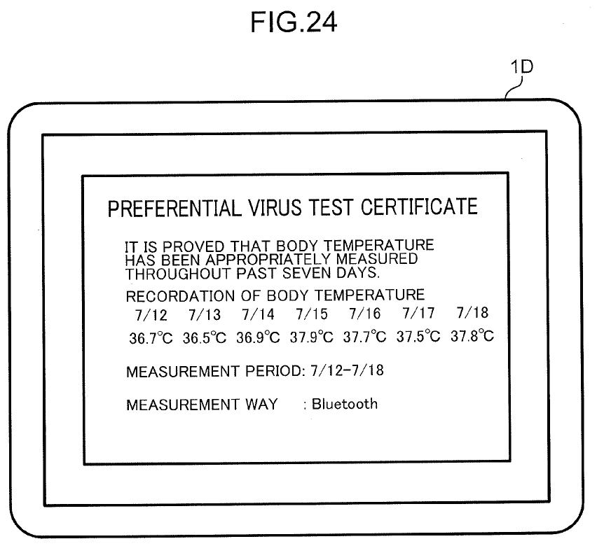

FIG. 24 shows an example of a preferential virus test certificate presentation screen image displayed on the first user terminal 1D in the fifth embodiment.

As shown in FIG. 24, the display control part 104D executes displaying of the preferential virus test certificate when the selected input mode continuously indicates the communication input mode throughout four days and the acquired body temperature information continuously indicates 37.5° C. or higher throughout the four days. The displayed preferential virus test certificate presentation screen image in FIG. 24 includes: a sentence confirming appropriate measurement of a body temperature throughout past seven days; body temperature values in the period of past seven days; a measurement period; and a measurement way. The measurement way shows acquisition of the body temperature information through the Bluetooth. The preferential virus test certificate presentation screen image in FIG. 24 shows the body temperature of 37.5° C. or higher throughout the past four days.

In the fifth embodiment, when the body temperature acquisition part 103 can acquire measurement device information, the preferential virus test certificate generation part 107 may generate a preferential virus test certificate including at least one of measurement of a body temperature in either a predictive measurement manner or an actual measurement manner, measurement of the body temperature in either a contact manner or a contactless manner, and a body portion at which the body temperature has been measured.

A preferential virus test certificate proving that the first user is preferentially prompted to receive a virus test is generated and displayed when a selected input mode continuously indicates the communication input mode throughout a predetermined period and acquired body temperature information continuously indicates a predetermined value or higher through a predetermined period as described above, and therefore, presentation of the necessity of preferentially prompting the first user to receive the virus test is achieved.

Here, the preferential virus test certificate generation part 107 may only determine whether the acquired body temperature information continuously indicates the predetermined value or higher throughout the predetermined period without determining whether the selected input mode continuously indicates the communication input mode throughout the predetermined period. In this case, the preferential virus test certificate generation part 107 may generate the preferential virus test certificate when the acquired body temperature information continuously indicates the predetermined value or higher throughout the predetermined period.

Furthermore, although the processor 11D of the first user terminal 1D realizes the preferential virus test certificate generation part 107 in the fifth embodiment, the disclosure is not limited thereto, and a processor included in the body temperature management server 3 may realize the preferential virus test certificate generation part 107. Specifically, the body temperature management server 3 may generate a preferential virus test certificate when a selected input mode continuously indicates the communication input mode throughout a predetermined period and acquired body temperature information continuously indicates a predetermined value or higher throughout a predetermined period. The body temperature management server 3 may transmit the generated preferential virus test certificate to the first user terminal 1D.

The input receiving part 13 may receive an input of health condition information by the first user when a body temperature is measured. The preferential virus test certificate generation part 107 may acquire the health condition information indicating a health condition of the first user from the input receiving part 13. The health condition information indicates the health condition of the first user, such as, coughing, feeling of fatigue, or goodness. The input receiving part 13 receives a selection of at least one item by the first user from among a plurality of items each showing a health condition. The items include, for example, goodness, coughing, feeling of fatigue, a sore throat, diarrhea, a headache, and a taste disorder. The preferential virus test certificate generation part 107 may generate a preferential virus test certificate including body temperature information, and health condition information in acquisition of the body temperature information.

Besides, the preferential virus test certificate generation part 107 may retrieve a hospital or clinic which can conduct a virus test among hospitals and clinics located in the vicinity of a residence of the first user. The display control part 104D may executes displaying of the hospital or clinic which can conduct the virus test together with the preferential virus test certificate.

In a modification of the fifth embodiment, a preferential virus test certificate generation part 107 may generate a preferential virus test certificate for each distinctive reliability level in accordance with an input mode and a measurement frequency.

The preferential virus test certificate generation part 107 may generate a first reliability level-preferential virus test certificate showing a highest reliability level when a measurement frequency in a predetermined period (e.g., seven days) indicates 100% (e.g., seven times of measurement respectively in the period of seven days), all the selected input modes indicate the communication input mode, and acquired body temperature information continuously indicates a predetermined value or higher throughout a predetermined period.

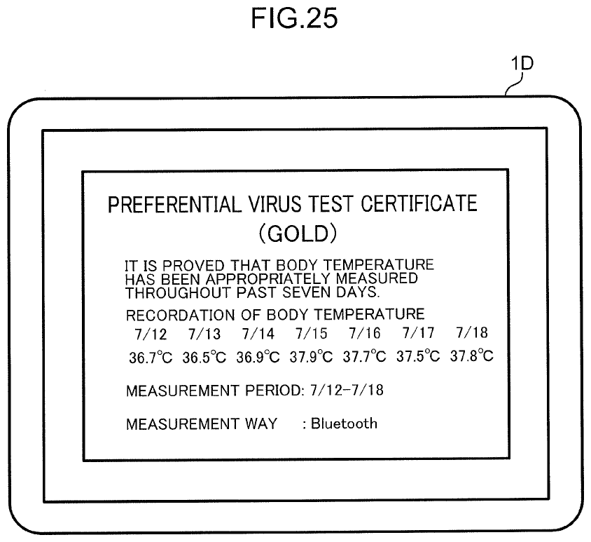

FIG. 25 shows an example of a first reliability level-preferential virus test certificate presentation screen image displayed on a first user terminal 1D in the modification of the fifth embodiment.

As shown in FIG. 25, a display control part 104D executes displaying of the first reliability level-preferential virus test certificate, when a measurement frequency in the period of seven days indicates 100%, all the selected input modes indicate the communication input mode, and the acquired body temperature information continuously indicates 37.5° C. or higher throughout four days. The displayed preferential virus test certificate presentation screen image in FIG. 25 includes: a reliability level; a sentence confirming appropriate measurement of a body temperature in the period of past seven days; body temperature values in the period of past seven days; a measurement period; and a measurement way. The reliability level is represented by one of gold, silver, and bronze. When the reliability level indicates the first reliability level, the preferential virus test certificate is displayed with “gold”. The measurement way shows acquisition of the body temperature information through the Bluetooth. The preferential virus test certificate presentation screen image in FIG. 25 shows the body temperature of 37.5° C. or higher throughout the past four days.

The preferential virus test certificate generation part 107 may generate a second reliability level-preferential virus test certificate showing a second highest reliability level when a measurement frequency in a predetermined period (e.g., seven days) indicates 80% or higher (e.g., six times of measurement in the period of seven days), all the selected input modes indicate the communication input mode, and acquired body temperature information continuously indicates a predetermined value or higher throughout a predetermined period.

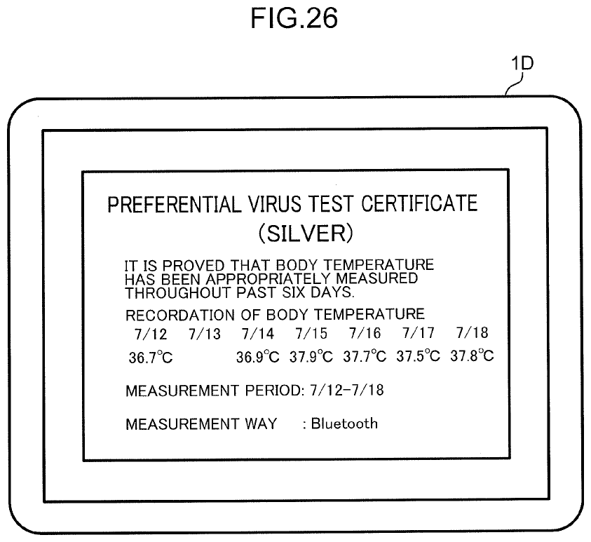

FIG. 26 shows an example of a second reliability level-preferential virus test certificate presentation screen image displayed on the first user terminal 1D in the modification of the fifth embodiment.

As shown in FIG. 26, the display control part 104D executes displaying of the second reliability level-preferential virus test certificate, when a measurement frequency in the period of seven days indicates 80% or higher (six times), all the selected input modes indicate the communication input mode, and the acquired body temperature information continuously indicates 37.5° C. or higher throughout four days. The displayed preferential virus test certificate presentation screen image in FIG. 26 includes: a reliability level; a sentence confirming appropriate measurement of a body temperature throughout past six days; body temperature values in the period of past six days; a measurement period; and a measurement way. The reliability level is represented by one of gold, silver, and bronze. When the reliability level indicates the second reliability level, the preferential virus test certificate is displayed with “silver”. The measurement way shows acquisition of the body temperature information through the Bluetooth. The preferential virus test certificate presentation screen image in FIG. 26 shows the body temperature of 37.5° C. or higher throughout the past four days.

The preferential virus test certificate generation part 107 may generate a third reliability level-preferential virus test certificate showing a third highest reliability level, when a measurement frequency in a predetermined period (e.g., seven days) indicates 50% or higher (e.g., four times of measurement in the period of seven days), all the selected input modes indicate the communication input mode, and acquired body temperature information continuously indicates a predetermined value or higher throughout a predetermined period. The preferential virus test certificate generation part 107 may further generate the third reliability level-preferential virus test certificate showing the third highest reliability level, when a measurement frequency in a predetermined period (e.g., seven days) indicates 80% or higher (e.g., six times of measurement in the period of seven days), not all the selected input modes indicate the communication input mode (i.e., the selected input modes include the manual input mode), and acquired body temperature information continuously indicates a predetermined value or higher throughout a predetermined period.

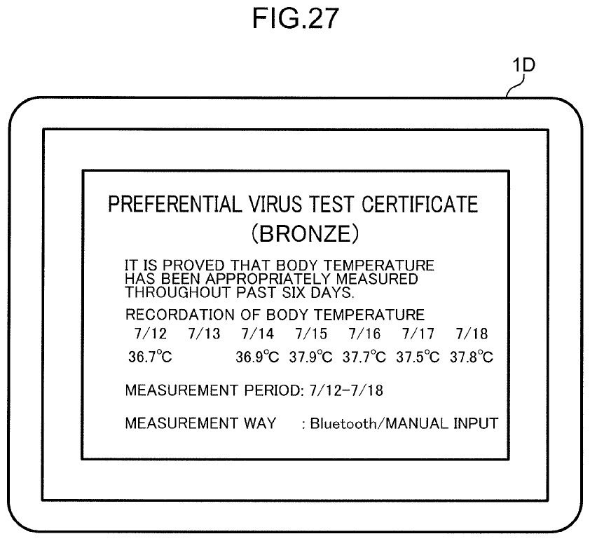

FIG. 27 shows an example of a third reliability level-preferential virus test certificate presentation screen image displayed on the first user terminal 1D in the modification of the fifth embodiment.

As shown in FIG. 27, the display control part 104D executes displaying of the third reliability level-preferential virus test certificate when a measurement frequency in a period of seven days indicates 80% or higher (six times), the selected input modes include the communication input mode and the manual input mode, and the acquired body temperature information continuously indicates 37.5° C. or higher throughout four days. The displayed preferential virus test certificate presentation screen image in FIG. 27 includes; a reliability level; a sentence confirming appropriate measurement of a body temperature in the period of past six days; body temperature values in the period of past six days; a measurement period; and a measurement way. The reliability level is represented by one of gold, silver, and bronze. When the reliability level indicates the third reliability level, the preferential virus test certificate is displayed with “bronze”. The measurement way shows acquisition of the body temperature information through the Bluetooth and manual input. The preferential virus test certificate presentation screen image in FIG. 27 shows the body temperature of 37.5° C. or higher throughout the past four days.

The first user terminal 1 may receive an input of vaccination information against a virus infection by the first user, and transmit the vaccination information against the virus infection to the body temperature management server 3. At this time, the display control part 202 of the second user terminal 2 may change an icon on the display part 24 in accordance with the vaccination information. In this manner, the second user can determine whether a change in a body temperature of the first user is caused by the relevant vaccination.

The body temperature information may be changed after being input. Specifically, the first user terminal 1 may receive a change in the input body temperature information. In this respect, when the body temperature is acquired from a device like a Bluetooth thermometer, the first user terminal 1 avoids changing the acquired body temperature information. Alternatively, in a case where a change in body temperature information acquired from a device like a Bluetooth thermometer is allowed and the body temperature information acquired from the device like the Bluetooth thermometer is changed, each of the first user terminal 1 and the second user terminal 2 avoids displaying an icon indicating the acquisition of the body temperature information in the communication input mode.

When the body temperature management server 3 does not receive a measured body temperature from the first user terminal 1 of the first user and receives a measured body temperature from another user terminal of another member in a group to which the first user belongs, the measurement of the body temperature by another member may be notified to the first user terminal 1. When the body temperature management server 3 does not receive a measured body temperature from the first user terminal 1 of the first user and a body temperature of another member in a group to which the first user belongs is continuously measured throughout a predetermined period, the continuous measurement of the body temperature by another member throughout the predetermined period may be notified to the first user terminal 1.

The body temperature management server 3 may calculate a temperature measurement rate of the group to which the first user belongs on an exact day, and transmits the calculated temperature measurement rate to the first user terminal 1.

When the body temperature management server 3 does not receive a measured body temperature from the first user terminal 1 of the first user and receives a measured temperature from a user terminal of another user whose action pattern of temperature measurement is similar to an action pattern of temperature measurement of the first user, the measurement of the body temperature by another user whose action pattern of temperature measurement is similar to the action pattern of temperature measurement of the first user may be notified to the first user terminal 1. Such another user whose action pattern of temperature measurement is similar to the action pattern of temperature measurement of the first user represents, for example, another user whose temperature measurement rate in a predetermined period is similar to a temperature measurement rate of the first user in the period, or another user whose temperature measurement time is similar to a temperature measurement time of the first user.

When the body temperature management server 3 does not receive a measured body temperature from the first user terminal 1 of the first user and the second user being a manager of a group to which the first user belongs confirms body temperature information about a certain member in the group, confirmation of the body temperature information about the member by the second user may be notified to the first user terminal 1.

The body temperature management server 3 may notify the first user terminal 1 of an optimal temperature measurement place for the first user and/or an optimal temperature measurement time for the first user. For instance, the body temperature management server 3 may detect an action pattern of the first user and give notification of a place where the first user stays longest in a period from waking up to going out as the optimal temperature measurement place for the first user. For instance, the body temperature management server 3 may give notification of a time period during which the first user stays in the optimal temperature measurement place for the first user as the optimal temperature measurement time for the first user.

The body temperature management server 3 may transmit alert information to the first user terminal 1 when body temperature information indicating a predetermined value or higher is continuously measured throughout a predetermined period. The predetermined value is, for example, 37.5° C., and the predetermined period includes, for example, four days.

The body temperature management server 3 may determine whether the first user has a fever, and transmit, to the first user terminal 1, a message recommending self-quarantine when the first user has a fever. The body temperature management server 3 may determine that the first user has a fever when the body temperature information about the first user indicates 37° C. or higher.

The body temperature management server 3 may store schedule information about a plurality of users working at the same company. When body temperature information about one user indicates 37° C. or higher among the body temperature information about the users, the body temperature management server 3 may transmit alert information to a user terminal of each user, other than the one user, who is to report to work.

The body temperature management server 3 may change a frequency of giving notification of a message encouraging measurement of a body temperature to the first user in accordance with implementation or non-implementation of a preventive measure against a virus spread (e.g., issuance or non-issuance of a declaration of a state of emergency from a government). Specifically, the body temperature management server 3 may set a frequency of notification of a message encouraging measurement of a body temperature to the first user in implementation of a preventive measure against a virus spread to be higher than a frequency of the notification of the message encouraging measurement of a body temperature to the first user in non-implementation of the preventive measure against the virus spread.

The body temperature management server 3 may change wording in a message encouraging measurement of a body temperature for the notification to the first user who has not measured a body temperature thereof in accordance with implementation or non-implementation of a preventive measure against a virus spread (e.g., issuance or non-issuance of a declaration of a state of emergency from a government). Specifically, the body temperature management server 3 may notify the first user of a message saying, “Make sure to measure your body temperature.”, in implementation of a preventive measure against a virus spread, and may notify the first user of a message saying, “Measure your body temperature.”, in non-implementation of the preventive measure against the virus spread.

The body temperature management system may further include a Web meeting server communicably connecting a plurality of user terminals to one another via a network to hold a web meeting by sharing images or pictures and voices acquired at the respective terminals. Each user terminal may receive an input of a body temperature of each user and a current health condition of each user, and transmit the input body temperature information and health condition information to the body temperature management server 3. The body temperature management server 3 may transmit the body temperature information and the health condition information received from each user terminal to the Web meeting server. When the Web meeting is held among a plurality of users, the Web meeting server may display, on a Web meeting screen image, an icon indicating a body temperature having a predetermined value or higher about a certain user or an icon indicating a symptom of a specific disease from a health condition of the user together with an image of the user. For instance, when the user has the body temperature of 37° C. or higher, the Web meeting server may display, on the Web meeting screen image, an icon indicating that the user has a fever together with an image of the user. Besides, for instance, when the health condition of the user indicates coughing, the Web meeting server may display, on the Web meeting screen image, an icon indicating the coughing of the user together with an image of the user. In this manner, each user can know a body temperature and a health condition of other user in the Web meeting.

In addition, the first user terminal 1 may receive an input of a residence of the first user and a current health condition of the first user, and transmit the input residence information and health condition information to the body temperature management server 3. When the health condition information about the first user shows a symptom of a specific disease, the body temperature management server 3 may specify a hospital or clinic suitable for treatment of the specific disease, and notify the first user terminal 1 of the specified hospital or clinic.

In each embodiment, each constituent element may be realized with dedicated hardware or by executing a software program suitable for the constituent element. Each constituent element may be realized by a program execution unit, such as a CPU or a processor, reading out and executing a software program recorded on a recording medium, such as a hard disk or a semiconductor memory. Other independent computer system may implement a program by recording the program in a recording medium to be transferred, or transferring the program via a network.

A part or all of functions of the device according to the embodiment of the disclosure are typically realized as a large scale integration (LSI), which is an integrated circuit. These functions may be formed as separate chips, or some or all of the functions may be included in one chip. The circuit integration is not limited to the LSI, and may be realized with a dedicated circuit or a general-purpose processor. A field programmable gate array (FPGA) that is programmable after manufacturing of an LSI or a reconfigurable processor in which connections and settings of circuit cells within the LSI are reconfigurable may be used.

A part or all of functions of the device according to the embodiment of the present disclosure may be implemented by a processor, such as a CPU executing a program.

Numerical values used above are merely illustrative to be used to specifically describe the present disclosure, and thus the present disclosure is not limited to the illustrative numerical values.

Order in which steps shown in the flowcharts are executed is merely illustrative to be used to specifically describe the present disclosure, and thus steps may be executed in order other than the above order as long as similar effects are obtained. Some of the steps may be executed simultaneously (in parallel) with other steps.

Industrial Applicability

The technology according to this disclosure achieves, when presenting measured body temperature information about a first user, presentation of reliability for the body temperature information to a second user, and therefore is useful as a technology of presenting the body temperature information about the first user to the second user.

1. An information processing method for an information processing system including a first terminal that acquires body temperature information about a first user and a second terminal that presents the body temperature information to a second user, the information processing method comprising:
causing the first terminal to:
select an input mode for use in acquiring the body temperature information from among a plurality of input modes each having distinctive reliability for body temperature information to he acquired;
acquire the body temperature information in the selected input mode; and
transmit the acquired body temperature information and input mode identification information indicating the selected input mode; and
causing the second terminal to:
receive the body temperature information and the input mode identification information; and
cause a display part to display the body temperature information in a distinctive display manner in accordance with the input mode identification information.

2. The information processing method according to claim 1, wherein the input modes include a first input mode of acquiring the body temperature information from a thermometer, which measures a body temperature of the first user, through communication therewith and a second input mode of acquiring the body temperature information from an input receiving part which receives a manual input of the body temperature by the user, and,
in the displaying of the body temperature information, the input mode indicated by the input mode identification information is determined as either the first input mode or the second input mode, and the body temperature information is displayed on the display part in a first display manner when the input mode is determined as the first input mode, and the body temperature information is displayed on the display part in a second display manner which is different from the first display manner when the input mode is determined as the second input mode.

3. The information processing method according to claim 2, comprising causing the first terminal to:
further generate a good health condition certificate proving that the first user has a good health condition when the selected input mode indicates the first input mode and the acquired body temperature information falls below a predetermined value; and
further cause a display part to display the generated good health condition certificate.

4. The information processing method according to claim 3, wherein,
in the generating of the good health condition certificate, the good health condition certificate is generated when the selected input mode continuously indicates the first input mode throughout a predetermined period and the acquired body temperature information continuously falls below the predetermined value throughout the predetermined period.

5. The information processing method according to claim 2, comprising causing the first terminal to:
further generate a preferential virus test certificate proving that the first user is preferentially prompted to receive a virus test when the selected input mode continuously indicates the first input mode throughout a predetermined period and the acquired body temperature information continuously indicates the predetermined value or higher throughout the predetermined period; and
cause the display part to further display the generated preferential virus test certificate.

6. The information processing method according to claim 1, wherein,
in the displaying of the body temperature information, a sign having a distinctive shape in accordance with the input mode identification information is displayed in the vicinity of the body temperature information, a sign having a distinctive color in accordance with the input mode identification information is displayed in the vicinity of the body temperature information, the body information is displayed in a distinctive color in accordance with the input mode identification information, or a name of the input mode indicated by the input mode identification information is displayed in the vicinity of the body temperature information.

7. The information processing method according to claim 1, wherein the first terminal causes the display part to display the body temperature information in a distinctive display manner in accordance with the selected input mode.

8. The information processing method according to claim 1, wherein,
in the acquiring of the body temperature information, measurement device information is acquired together with the body temperature information, the measurement device information including at least one of information indicating whether a body temperature is measured in a predictive measurement manner or an actual measurement manner, information indicating whether the body temperature is measured in a contact manner or a contactless manner, and measured portion information indicating a body portion at which the body temperature has been measured,
in the transmitting of the body temperature information and the input mode identification information, the acquired measurement device information is transmitted together with the body temperature information and the input mode identification information,
in the receiving of the body temperature information and the input mode identification information, the measurement device information is received together with the body temperature information and the input mode identification information, and
in the displaying of the body temperature information, the body temperature information is displayed on the display part in a distinctive display manner in accordance with the input mode identification information and the measurement device information.

9. The information processing method according to claim 1, wherein,
in the acquiring of the body temperature information, device identification information for identifying a thermometer which measures a body temperature of the first user is acquired together with the body temperature information,
in the transmitting of the body temperature information and the input mode identification information, the acquired device identification information is transmitted together with the body temperature information and the input mode identification information,
in the receiving of the body temperature information and the input mode identification information, measurement device information is received together with the body temperature information and the input mode identification information, the measurement device information being specified on the basis of the device identification information and including at least one of information indicating whether the body temperature is measured in a predictive measurement manner or an actual measurement manner, information indicating whether the body temperature is measured in a contact manner or a contactless manner, and measured portion information indicating a body portion at which the body temperature has been measured, and
in the displaying of the body temperature information, the body temperature information is displayed on the display part in a distinctive display manner in accordance with the input mode identification information and the measurement device information.

10. An information processing system, comprising:
a first terminal that acquires body temperature information about a first user; and
a second terminal that presents the body temperature information to a second user, wherein
the first terminal includes:
a selection part that selects an input mode for use in acquiring the body temperature information from among a plurality of input modes each having distinctive reliability for body temperature information to be acquired;
an acquisition part that acquires the body temperature information in the selected input mode; and
a transmission part that transmits the acquired body temperature information and input mode identification information indicating the selected input mode, and
the second terminal includes:
a receiving part that receives the body temperature information and the input mode identification information; and
a display control part that causes a display part to display the body temperature information in a distinctive display manner in accordance with the input mode identification information.

11. An information processing method for a second terminal that presents body temperature information about a first user acquired by a first terminal to a second user, the information processing method comprising:
receiving, from the first terminal which: selects an input mode for use in acquiring the body temperature information from among a plurality of input modes each having distinctive reliability for body temperature information to be acquired; acquires the body temperature information in the selected input mode; and transmits the acquired body temperature information and input mode identification information indicating the input mode, the body temperature information and the input mode identification information; and
causing a display part to display the body temperature information in a distinctive display manner in accordance with the input mode identification information.

12. A terminal that presents body temperature information about a first user acquired by another terminal to a second user, the terminal comprising:
a receiving part that receives, from the another terminal which: selects an input mode for use in acquiring the body temperature information from among a plurality of input modes each having distinctive reliability for body temperature information to be acquired; acquires the body temperature information in accordance with the selected input mode; and transmits the acquired body temperature information and input mode identification information indicating the selected input mode, the body temperature information and the input mode identification information; and
a display control part that causes a display part to display the body temperature information in a distinctive display manner in accordance with the input mode identification information.

13. A non-transitory computer readable recording medium storing an information processing program for presenting body temperature information about a first user acquired by another terminal to a second user, the information processing program comprising:
causing a computer to execute:
receiving, from the another terminal which: selects an input mode for use in acquiring the body temperature information from among a plurality of input modes each having distinctive reliability for body temperature information to be acquired; acquires the body temperature information in the selected input mode; and transmits the acquired body temperature information and input mode identification information indicating the selected input mode, the body temperature information and the input mode identification information; and
causing a display part to display the body temperature information in a distinctive display manner in accordance with the input mode identification information.
